(19) United States
(12) Patent Application Publication
(10) Pub. No.: US 2023/0218251 A1
(43) Pub. Date: Jul. 13, 2023

(54) MINI C-ARM WITH MOVABLE SOURCE

(71) Applicant: HOLOGIC, INC., Marlborough, MA (US)

(72) Inventors: Marc HANSROUL, Marlborough, MA (US); Daniel SANTOS, Marlborough, MA (US); Jay STEIN, Marlborough, MA (US); Tri PHAM, Marlborough, MA (US); Scott HUNT, Marlborough, MA (US)

(73) Assignee: HOLOGIC, INC., Marlborough, MA (US)

(21) Appl. No.: 18/001,409

(22) PCT Filed: Jun. 9, 2021
(86) PCT No.: PCT/US2021/036611

§ 371 Date: Dec. 9, 2022

Related U.S. Application Data
(60) Provisional application No. 63/037,263, filed on Jun. 10, 2020.

(51) Int. Cl. A61B 6/00; A61B 6/02

(52) U.S. Cl. CPC A61B 6/4452; A61B 6/025; A61B 6/4405; A61B 6/4441; A61B 6/4476; A61B 6/5205; A61B 6/463; A61B 6/466; A61B 6/54; A61B 6/447

(57) ABSTRACT

A mini C-arm with a movable X-ray source is disclosed. The mini C-arm including a moveable base, a C-arm assembly, and an arm assembly for coupling the C-arm assembly and the base. The C-arm assembly includes a first end, a second end, and a curved intermediate body portion defining an arc length. The source is positioned adjacent to the first end. A detector is positioned at the second end. The source is moveable along the arc length and relative to the detector to enable a plurality of images of the patient's anatomy to be acquired including a first image when the X-ray source is at a first position and a second image when the X-ray source is at a second position. The images being taken without moving the patient's anatomy. The C-arm assembly may include a motor and a belt drive system for moving the source relative to the detector.

### CROSS-REFERENCE TO RELATED APPLICATIONS

This is a non-provisional of, and claims the benefit of the filing date of, pending U.S. provisional patent application No. 63/037,263, filed Jun. 10, 2020, entitled “Mini C-arm with Movable Source and/or Detector,” the entirety of which application is incorporated by reference herein.

### FIELD OF THE DISCLOSURE

The present invention generally relates to imaging systems, and, more particularly, to a mobile imaging system such as, for example, a mini C-arm having a movable X-ray source.

### BACKGROUND OF THE DISCLOSURE

Mini C-arms are mobile X-ray fluoroscopic imaging systems that provide non-invasive means for imaging a patient's bone and/or tissue (collectively a patient's anatomy). These systems are used by orthopedic surgeons during surgery on extremities (e.g., hand, wrist, elbow, leg, foot, ankle, etc.) to evaluate the patient's anatomy and guide procedures where various internal and/or external hardware devices such as, for example, bone plates, screw, pins, wires, etc. (collectively referred to herein as orthopedic devices without the intent to limit) are used. For example, surgeons may acquire X-ray images during a surgery to repair a fractured bone in order to visualize the anatomy and confirm the position and orientation of the orthopedic devices used to fix and stabilize the fracture.

Conventional mini C-arms have an X-ray source that is in a fixed relationship relative to an X-ray detector. The X-ray source and detector are mounted on opposing ends of a one-piece support assembly having a substantially “C” or “U” shape (referred to herein as a C-arm assembly). The imaging components are aligned on an imaging axis and have a fixed X-ray source to image detector distance (SID). This arrangement can present certain limitations. That is, in connection with mini C-arms, the detector's maximum distance between the X-ray source and detector or SID is fixed and cannot be exceed. For example, generally speaking, conventional mini C-arms include fixed imaging components (e.g., X-ray source and detector), which are located a fixed distance from each other (e.g., a fixed SID equal to or less than 45 cm).

The detector is often used as an operating table during orthopedic surgical procedures. Once a patient's anatomy is placed on the detector, the surgeon is unable to move the C-arm assembly. In certain instances, it is desirable to obtain multiple X-ray views or projections of a patient's anatomy. For example, a surgeon may want to acquire multiple X-ray views (e.g., anterior-posterior view, oblique view, lateral view, etc.) during a bone fracture procedure to, for example, assess the depth, position and/or angle of the surgical tool (e.g., drill) used to place the orthopedic devices. Additionally, the surgeon may want to confirm the position of the orthopedic devices after they have been inserted into or secured to the patient's anatomy. With conventional mini C-arms, surgeons may acquire those views by removing the patient's anatomy from the detector surface and repositioning the C-arm assembly or by changing the position of the patient's anatomy relative to the X-ray source and detector. Depending on the surgical procedure and type of orthopedic devices involved, having to move the patient's anatomy may add risk to the procedure and may be undesirable.

It is with respect to these and other considerations that the present improvements may be useful.

### SUMMARY OF THE DISCLOSURE

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

In one embodiment, a mini C-arm imaging apparatus is disclosed. The mini C-arm imaging apparatus comprising a C-arm assembly, a movable base, and an arm assembly coupling the C-arm assembly to the movable base. The C-arm assembly includes a first end, a second end, and a curved intermediate body portion extending between the first and second ends. The C-arm assembly also includes an X-ray source adjacent the first end and a detector at the second end. The curved intermediate body portion defines an arc length extending between the first and second ends. The X-ray source being moveable along the arc length of the curved intermediate body portion and relative to the detector to enable the mini C-arm to acquire a first image when the X-ray source is at a first position on the curved intermediate body portion and a second image when the X-ray source is at a second position on the curved intermediate body portion, the second position being different that the first position, so that the first and second images of the patient's anatomy are taken at different angles relative to the patient's anatomy and are acquired without moving the patient's anatomy during a surgical procedure.

In one embodiment, the curved intermediate body portion of the C-arm assembly includes a rail, the X-ray source being movably coupled to the rail.

In one embodiment, the X-ray source is manually movable along a length of the rail.

In one embodiment, the X-ray source is moved along a length of the rail via a drive system. In one embodiment, the drive system includes a motor operatively coupled to a belt and one or more idlers, and wherein activation of the motor rotates the belt about the one or more idlers to move the X-ray source along the length of the rail.

In one embodiment, the X-ray source includes a connector unit movably coupled to the rail and a directional alignment feature for guiding movement along the length of the rail.

In one embodiment, the mini C-arm imaging apparatus further comprises a dynamic counterweight to balance the X-ray source as the X-ray source moves along the length of the rail.

In one embodiment, the C-arm assembly further comprises an intermediate link member coupled to the curved intermediate body portion adjacent the first end of the C-arm assembly, wherein the X-ray source is movable coupled to the intermediate link member to position the X-ray source along the arc length of the curved intermediate body portion. In one embodiment, the intermediate link member is fixed to the C-arm assembly. In one embodiment, the intermediate link member is movably coupled to the C-arm assembly.

In one embodiment, the X-ray source moves ±20 degrees along the arc length of the curved intermediate body portion of the C-arm assembly and relative to an axis passing through the X-ray source and the detector when the X-ray source is positioned directly above the detector.

In one embodiment, the detector is rotatable about an axis passing through the X-ray source and the detector when the X-ray source is positioned directly above the detector. In one embodiment, the detector is positioned within a housing, the housing is rotatably coupled to the second end of the curved intermediate body portion of the C-arm assembly.

In one embodiment, the X-ray source is movable along an arc extending perpendicular to the arc length of the curved intermediate body portion of the C-arm assembly. In one embodiment, the X-ray source is positioned within a source housing, the source housing and the X-ray source are movable relative to the detector along the arc extending perpendicular to the arc length of the curved intermediate body portion of the of the C-arm assembly. In one embodiment, the X-ray source is positioned within a source housing, the X-ray source is movable relative to the source housing and the detector along the arc extending perpendicular to the arc length of the curved intermediate body portion of the of the C-arm assembly.

In one embodiment, the mini C-arm imaging apparatus further comprises a secondary link member, the secondary link member includes a first end rotatably coupled to the C-arm assembly and a second end coupled to the X-ray source, the secondary link member being rotatable relative to the C-arm assembly so that the X-ray source moves along the arc extending perpendicular to the arc length of the curved intermediate body portion of the of the C-arm assembly.

In one embodiment, a mini C-arm imaging apparatus is disclosed. The mini C-arm imaging apparatus comprises a C-arm assembly, a movable base, and an arm assembly coupling the C-arm assembly to the movable base. The C-arm assembly includes a first end, a second end, a curved intermediate body portion extending between the first and second ends, and a rail coupled to the C-arm assembly and extending between portions of the curved intermediate body portion of the C-arm assembly. The rail defines an arc length. An X-ray source is movably coupled to the rail. A detector is positioned at the second end of the C-arm assembly and a drive system is associated with the X-ray source, the drive system including a motor operatively coupled to a belt and one or more idlers, wherein activation of the motor rotates the belt about the one or more idlers to move the X-ray source along the arc length of the rail.

In one embodiment, the X-ray source is movable along the arc length of the rail to enable the mini C-arm to acquire a first image at a first position along the curved intermediate portion and a second image at a second position along the curved intermediate portion, the second position being different that the first position so that first and second images of the patient's anatomy are taken at different angles and are acquired without moving the patient's anatomy during a surgical procedure.

In one embodiment, the X-ray source includes a connector unit movably coupled to the rail and a directional alignment feature for guiding movement along the arc length of the rail.

In one embodiment, the X-ray source provides ±20 degrees of movement relative to the detector and an imaging axis along the arc length of the rail, the imaging axis being defined as the axis passing through the X-ray source and the detector when the X-ray source is positioned directly above the detector.

In one embodiment, the detector is rotatable about an axis passing perpendicular to a surface of the detector.

In one embodiment, the mini C-arm imaging apparatus further comprises a motion control system to control movement of the x-ray source along the arc length of the rail.

In one embodiment, a method of acquiring multiple images using a mini C-arm is disclosed. The mini C-arm includes a C-arm assembly having a first end, a second end, a curved intermediate body portion extending between the first and second ends, the mini C-arm including an X-ray source moveable along an arc length of the curved intermediate body portion of the C-arm assembly and a detector positioned at the second end of the C-arm assembly. The method comprises moving the X-ray source along the arc length of the curved intermediate body portion of the C-arm assembly relative to the detector between a first position on the curved intermediate body portion and a second position on the curved intermediate body portion and acquiring a plurality of projection images of a patient's anatomy without moving the patient's anatomy from a surface of the detector as the x-ray source moves between the first and second positions.

In one embodiment, the method further comprises displaying two or more projection images on a display device.

In one embodiment, the step of displaying the two or more projection images includes displaying the projection image acquired at the first position and the projection image acquired at the second position.

In one embodiment, the step of displaying the two or more projection images includes the step of selecting at least two projection images from the plurality of projection images acquired as the X-ray source moves between the first and second positions.

In one embodiment, the method further comprises displaying the two or more projection images with a video of all of the plurality of projection images acquired as the X-ray source moves between the first position and the second position.

In one embodiment, the method further comprises generating a three-dimensional reconstruction of the patient's anatomy using the plurality of projection images.

In one embodiment, the method further comprises displaying the three-dimensional reconstruction of the patient's anatomy.

In one embodiment, the method further comprises selecting one of multi-angle view (MAV) imagine acquisition mode or tomosynthesis (TOMO) image acquisition mode before acquiring the plurality of projection images; and processing the plurality of projection images for display on a display device based on the selected mode.

In one embodiment, the images are continuously acquired as the X-ray source moves between the first and second positions.

In one embodiment, the X-ray source automatically moves between the first and second positions.

### BRIEF DESCRIPTION OF THE DRAWINGS

By way of example, a specific embodiment of the disclosed device will now be described, with reference to the accompanying drawings, in which:

FIG. 1 is a perspective view of a conventional mobile imaging system or mini C-arm;

FIG. 2 is a perspective view of an example embodiment of a C-arm assembly in accordance with one or more features of the present disclosure, the C-arm assembly may be used in connection with the mini C-arm shown in FIG. 1;

FIG. 3 is a perspective view of the example embodiment of the C-arm assembly shown in FIG. 2 having a rotatable detector, and includes example images of a patient's anatomy at a posterior-anterior (AP) angle and in an oblique angle;

FIG. 4 is a side view of an example embodiment of the C-arm assembly shown in FIG. 2, in accordance with one or more features of the present disclosure, the C-arm assembly may be used in connection with the mini C-arm shown in FIG. 1;

FIGS. 5A-5D are various views of an example embodiment of the C-arm assembly shown in FIG. 4 in accordance with one or more features of the present disclosure, the C-arm assembly may be used in connection with the mini C-arm shown in FIG. 1;

FIG. 6 is a schematic view of an alternate drive system in accordance with one or more features of the present disclosure, the drive system may be used in connection with the C-arm assembly shown in FIGS. 5A-5D;

FIG. 7 is a schematic view of an alternate drive system in accordance with one or more features of the present disclosure, the drive system may be used in connection with the C-arm assembly shown in FIGS. 5A-5D;

FIG. 8 is a schematic view of an alternate drive system in accordance with one or more features of the present disclosure, the drive system may be used in connection with the C-arm assembly shown in FIGS. 5A-5D;

FIG. 9 illustrates various views of an alternate example embodiment of the C-arm assembly shown in FIG. 2, in accordance with one or more features of the present disclosure, the C-arm assembly may be used in connection with the mini C-arm shown in FIG. 1;

FIG. 10 illustrates various views of an alternate example embodiment of the C-arm assembly shown in FIG. 2, in accordance with one or more features of the present disclosure, the C-arm assembly may be used in connection with the mini C-arm shown in FIG. 1;

FIG. 11 is a schematic view of an alternate position sensing system in accordance with one or more features of the present disclosure, the position sensing system may be used in connection with the C-arm assemblies disclosed herein;

FIG. 12 is a schematic view of an alternate position sensing system in accordance with one or more features of the present disclosure, the position sensing system may be used in connection with the C-arm assemblies disclosed herein;

FIG. 13A is a front view of an alternate example embodiment of a C-arm assembly in accordance with one or more features of the present disclosure, the C-arm assembly may be used in connection with the mini C-arm shown in FIG. 1;

FIG. 13B is a front view of an alternate example embodiment of a C-arm assembly in accordance with one or more features of the present disclosure, the C-arm assembly may be used in connection with the mini C-arm shown in FIG. 1;

FIG. 14A is a side view of an alternate example embodiment of a C-arm assembly in accordance with one or more features of the present disclosure, the C-arm assembly may be used in connection with the mini C-arm shown in FIG. 1;

FIG. 14B is a front view of the C-arm assembly shown in FIG. 14A;

FIG. 15 is a perspective view of an alternate example embodiment of a C-arm assembly in accordance with one or more features of the present disclosure, the C-arm assembly may be used in connection with the mini C-arm shown in FIG. 1;

FIG. 16 is a perspective view of an alternate example embodiment of a C-arm assembly in accordance with one or more features of the present disclosure, the C-arm assembly may be used in connection with the mini C-arm shown in FIG. 1;

FIG. 17 is a perspective view of an alternate example embodiment of a C-arm assembly in accordance with one or more features of the present disclosure, the C-arm assembly may be used in connection with the mini C-arm shown in FIG. 1;

FIG. 18 is a flowchart of an example embodiment of an image acquisition method in accordance with one or more features of the present disclosure, the image acquisition method may be used in connection with the mini C-arms shown herein; and

FIG. 19 is a flowchart of an example embodiment of an image processing method in accordance with one or more features of the present disclosure, the image processing method may be used in connection with the mini C-arms shown herein.

The drawings are not necessarily to scale. The drawings are merely representations, not intended to portray specific parameters of the disclosure. The drawings are intended to depict example embodiments of the disclosure, and therefore are not be considered as limiting in scope. In the drawings, like numbering represents like elements unless otherwise noted.

### DETAILED DESCRIPTION

The present disclosure generally relates to mini C-arms, which are mobile X-ray fluoroscopic imaging systems, and methods of operating or controlling such systems. Numerous embodiments of a mini C-arm in accordance with the present disclosure are described hereinafter with reference to the accompanying drawings, in which preferred embodiments of the present disclosure are presented. The mini C-arm of the present disclosure may, however, be embodied in many different forms and should not be construed as being limited to the embodiments set forth herein. Rather, these embodiments are provided so that this disclosure will convey certain example features of the mini C-arm to those skilled in the art.

Mini C-arms are used for a wide range of orthopedic procedures including to image patient extremities and perform interventions. As an example, during a surgical procedure to set a fracture, the bone fragments are first repositioned (reduced) into their normal alignment and then held together with orthopedic devices, such as plates, screws, nails, and wires, etc. Surgeons may use the mini C-arm to image the patient's anatomy during these procedures. In certain instances, it may be desirable to obtain multiple X-ray views of a patient's anatomy to, for example, assess the position, depth, and/or angle of the surgical tools used to drill holes in the bone to insert or otherwise secure the orthopedic devices to the bone. Without such guidance, surgeons may have to remove the orthopedic devices from the bone to correct the position of these devices. It may also be desirable to obtain multiple X-ray views to confirm the placement of the orthopedic devices relative to the patient's anatomy after they have been inserted into or otherwise secured to the patient's anatomy.

As mentioned above, conventional mini C-arms have an X-ray source and a detector mounted on opposing ends of a C-arm assembly and fixed relative to each other and the C-arm assembly. As a result, while operators can move the C-arm assembly and the imaging components relative to the patient's anatomy to acquire images of the patient's anatomy at different angles, this requires removing the patient's anatomy from the detector and repositioning the imaging components relative to the patient and/or by changing the position of the patient's anatomy relative to the X-ray source and detector. These methods, which require moving the patient's anatomy, are undesirable particularly when performing surgeries to fix a fracture.

In accordance with one or more features of the present disclosure, as will be described in greater detail below, the mini C-arm includes a C-arm assembly including an X-ray source and a detector, a movable or mobile base or the like, and an arm assembly for coupling the C-arm assembly and the movable base. The X-ray source of the present disclosure is moveable relative to the C-arm assembly and the detector during a procedure to enable the surgeon to acquire multiple X-ray images at different positions and/or angles without moving the patient's anatomy. As an example, X-rays images can be acquired at different angles during a drilling procedure to provide information on the position or depth of the orthopedic devices to be placed in the patient's anatomy. This may allow surgeons to correct their position, insertion angle, depth, etc. of the drilling tool and/or the placement of the orthopedic devices in real-time. This has the benefits of reducing the likelihood of a second surgery, reducing risk of post-operative complications, reducing the procedure time by improving the workflow, and improving the overall quality of the procedure.

In one embodiment, the X-ray source or X-ray source module (terms used interchangeably without the intent to limit or distinguish) is mechanically coupled to the C-arm assembly and movable along an arc length of the C-arm assembly. The arc length may comprise a portion of or the entire curvature of the C-arm assembly. In certain additional embodiments, the detector may be rotatable about an axis passing perpendicular to a face of the detector. In alternative embodiments, the source is mechanically coupled to and movable along an arc perpendicular to the arc length of the C-arm assembly.

In accordance with one or more features of the present disclosure, and as will be described in greater detail herein, by enabling the X-ray source or X-ray source module to move relative to the detector, the mini C-arm enables multi-angle view (MAV) and/or tomosynthesis (TOMO) image acquisition. MAV and TOMO imaging acquisition methods involve acquiring fluoroscopic images of the patient's static anatomy while the angle of the X-ray beam from the source to the image plane of the detector is varied (e.g., the angle between the X-ray source beam and the detector image plane may be varied while the center of the X-ray source beam remains aligned with the center of the detector's image plane throughout the range of relative movement between the x-ray source and the detector). With TOMO, the X-ray source moves in an arc over the detector through a limited angle range to capture multiple images of the patient's anatomy from different angles. TOMO image acquisition may involve continuous acquisition over the angle range, which can be, for example, forty degrees (e.g., ±20 degrees from a center of the arc length of the intermediate body portion of the C-arm assembly or relative to imaging axis, e.g., axis passing thru the X-ray source and detector when the X-ray source is aligned directly over the detector, as will be described in greater detail herein), with exposures made every 1 degree or so during the scan. These images are then reconstructed or “synthesized” into a set of three-dimensional images by a computer. With MAV image acquisition, the X-ray source is movable to acquire two or more images including off-axis views of the patient's anatomy (e.g., an oblique view or a lateral view).

In certain embodiments, MAV image acquisition and TOMO image acquisition may utilize substantially the same process. That is, as will be described in greater detail herein, the mini C-arm enables a plurality of images at various views, projections, angles, etc. to be acquired. However, the image processing and display may differ between the two modes (e.g., MAV image acquisition mode and TOMO image acquisition mode). For example, in connection with MAV, the images may be displayed side-by-side illustrating two separate 2D images acquired at different angles. Meanwhile, with TOMO, a 3D reconstructed image may be generated and then displayed. Both MAV and TOMO may also display the full sequence of images acquired (e.g., 2D Cine-type image).

In either event, in order to acquire multiple angles or views of the patient's anatomy without moving the patient's anatomy (e.g., it is preferred to maintain the patient's anatomy static in relationship to the detector as images are acquired to reduce motion-blur imaging effects), it is preferable to move the X-ray source relative to the patient's anatomy and/or the detector during the image acquisition workflow. For mini C-arms, the distance from the X-ray source to the detector's image plane (SID) cannot exceed 45 cm. As such, the SID needs to be controlled as the X-ray source moves through its MAV/TOMO angle ranges (e.g., distance can vary slightly with limited compromise to image quality). That is, during movement of the X-ray source, control over the source movement must be controlled to maintain the SID (e.g., precise control over the X-ray source movement is desirable to control the SID so it does not exceed 45 cm).

With this in mind, the X-ray source of the present disclosure moves or rotates along an arc length that is centered at or about the top surface of the detector at the center of its active area (referred to hereinafter as the detector's image plane). In certain embodiments, the arc length may be equivalent to arc radius which, in turn, may be equivalent to the SID, e.g., 45 cm. However, it is envisioned that the arc radius may not be limited to 45 cm. For example, it is envisioned that the C-arm may allow for variable source to detector distances, where the SID does not exceed 45 cm. In these embodiments, the source may move along a larger or smaller arc length.

In order to achieve and control movement of the source along the arc length of the C-arm assembly, the mini C-arm preferably includes one or more of the following features: a mechanical travel path along the aforementioned arc length; a drive system such as, for example, a motorized drive subsystem to apply a force to the X-ray source to move the source along the travel path/arc length, and a motion control system to control the motion of the X-ray source. The motion control system may include one or more of the following features: a positioning sensing subsystem to measure the angular position of the X-ray source relative to the detector; an over-travel sensing subsystem to detect and limit the maximum range of travel of the X-ray source; and a collision-detection subsystem to detect and prevent the X-ray source from contacting an obstacle during its normal range of motion.

As will be described in greater detail herein, the C-arm assembly includes a mechanical travel path, which may be provided in the form of a track or a rail. The X-ray source module may include means for coupling to and moving along the track or rail. The track may be formed as an integral part of the intermediate body portion of the C-arm assembly or comprise a separate piece attached to the intermediate body portion of the C-arm assembly. The source may be directly or indirectly coupled to the track or rail so that the source can be moved, repositioned, etc. along the track or rail, which extends along the arc length A_{L} of the intermediate body portion of the C-arm assembly.

A force may be applied to the source module via, for example, a motorized drive subsystem to enable movement of the source along the arc length (e.g., the motorized drive subsystem applies a force to the X-ray source to move the X-ray source along the mechanical travel path (e.g., track or rail)). In one embodiment, the drive system may include a motor attached to a drive mechanism such as, for example, a lead screw, a belt-drive system, etc. In addition, the motor may contain a braking mechanism, e.g., a spring-assisted breaking mechanism, to lock the position of the X-ray source module when the motor is not in motion.

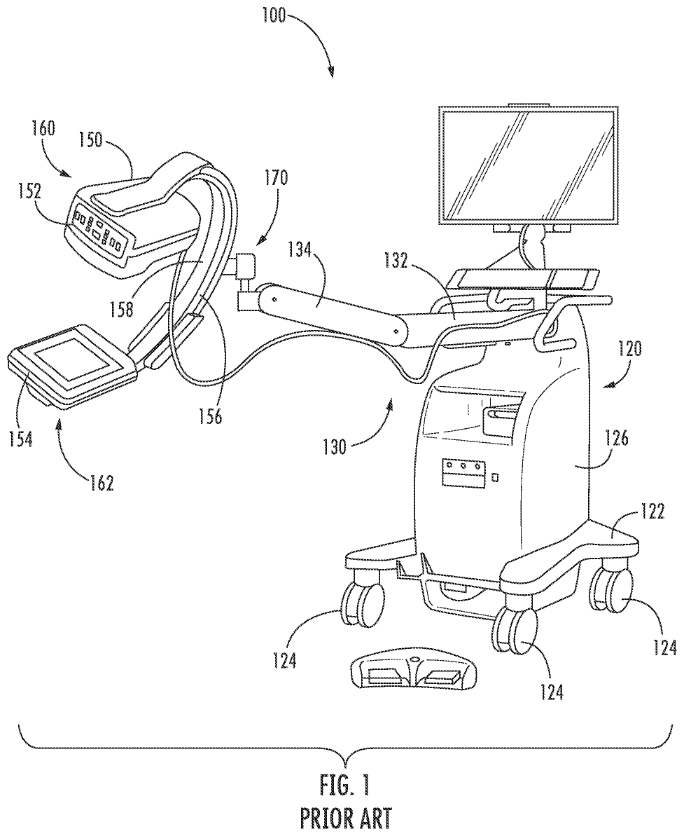

Referring now to FIG. 1, a convention embodiment of a mini C-arm 100 is shown. As illustrated, the mini C-arm 100 includes a base 120, a C-arm assembly 150, and an arm assembly 130 for coupling the C-arm assembly 150 to the base 120. As illustrated, the base 120 may include a platform 122 and a plurality of wheels 124 extending from a bottom surface of the platform 122 so that the base 120, and hence the mini C-arm 100, can be movably located by the operator as desired. The wheels 124 are selectably lockable by the user so that when in a locked state, the wheels 124 allow the operator to manipulate the arm assembly 130 without shifting the location or orientation of the base 120. The base 120 may also include a cabinet 126. As will be appreciated by one of ordinary skill in the art, the cabinet 126 may store, for example, controls (not shown) for operating the mini C-arm 100, electrical components (not shown) needed for operation of the mini C-arm 100, counterweights (not shown) needed to balance extension of the C-arm assembly 150, a brake system, a cord wrap, etc. The cabinet 126 may also include, for example, a keyboard, one or more monitors, a printer, etc.

Referring to FIG. 1, the arm assembly 130 may include a first arm 132 and a second arm 134, although it is envisioned that the arm assembly 130 may include a lesser or greater number of arms such as, for example, one, three, four, etc. The arm assembly 130 enables variable placement of the C-arm assembly 150 relative to the base 120. In one embodiment, the arm assembly 130, and more specifically the first arm 132, may be coupled to the base 120 via a vertically adjustable connection, although other mechanisms for coupling the arm assembly 130 to the base 120 are envisioned including, for example, a pivotable connection mechanism. The second arm 134 may be coupled to the first arm 132 via a joint assembly to enable the second arm 134 to move relative to the first arm 132. In addition, the second arm 134 may be coupled to the C-arm assembly 150 via an orbital mount 170, as will be described in greater detail below. Thus arranged, the arm assembly 130 enables the C-arm assembly 150 to be movably positioned relative to the base 120.

As will be appreciated by one of ordinary skill in the art, the mini C-arm 100 of the present disclosure may be used with any suitable base 120 and/or arm assembly 130 now known or hereafter developed. As such, additional details regarding construction, operation, etc. of the base 120 and/or the arm assembly 130 are omitted for sake of brevity of the present disclosure. In this regard, it should be understood that the present disclosure should not be limited to the details of the base 120 and/or arm assembly 130 disclosed and illustrated herein unless specifically claimed and that any suitable base 120 and/or arm assembly 130 can be used in connection with the principles of the present disclosure.

Referring to FIG. 1, and as previously mentioned, the mini C-arm 100 also includes a C-arm assembly 150. The C-arm assembly 150 includes a source 152, a detector 154, and an intermediate body portion 156 for coupling to the source 152 and the detector 154. As will be readily known by one of ordinary skill in the art, the imaging components (e.g., X-ray source 152 and detector 154) receive photons, convert the photons/X-rays to a manipulable electrical signal that is transmitted to an image processing unit (not shown). The image processing unit may be any suitable hardware and/or software system, now known or hereafter developed to receive the electrical signal and to convert the electrical signal into an image. Next, the image may be displayed on a monitor or TV screen. The image can also be stored, printed, etc. The image may be a single image or a plurality of images.

The intermediate body portion 156 of the C-arm assembly 150 includes a curved or arcuate configuration. For example, the intermediate body portion 156 may have a substantially “C” or “U” shape, although other shapes are envisioned. The intermediate body portion 156 may be a one-piece structure that includes a body portion 158 and first and second end portions 160, 162 for coupling to the source and detector 152, 154, respectively. Additionally, the C-arm assembly 150 may include an orbital mount 170 for coupling to the arm assembly 130. The orbital mount 170 may be coupled to the body portion 158 of the intermediate body portion 156. With this arrangement, the body portion 158, and hence the source and detector 152, 154, can rotate or orbit relative to the orbital mount 170 so that the operator is provided with increased versatility in positioning the imaging components relative to the patient's anatomy. As illustrated, the source 152 and the detector 154 are positioned at the first and second ends 160, 162 of the C-arm assembly 150 in facing relationship with each other.

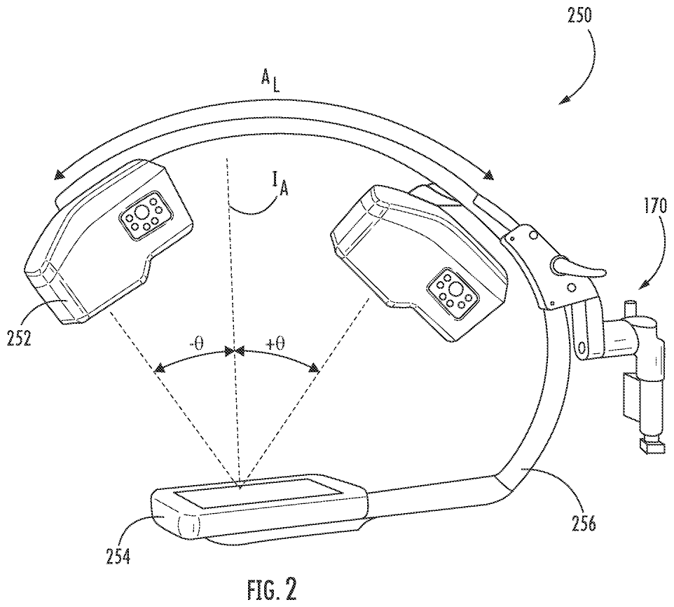

In contrast to conventional mini C-arms such as, for example, mini C-arm 100 shown in FIG. 1, wherein the source 152 and the detector 154 are fixedly coupled to the first and second ends 160, 162 of the C-arm assembly 150, in accordance with one or more features of the present disclosure, the source moves or rotates about an imaging axis extending through the center of the detector. Referring to FIG. 2, in one example embodiment in accordance with the present disclosure, the mini C-arm may include a C-arm assembly 250 including a source 252, a detector 254, and an intermediate body portion 256 wherein the source 252 moves along the curvature of the intermediate body portion 256 of the C-arm assembly 250. In this example, the source 252 can move along a portion of the arc length A_{L} of the intermediate body portion 256, however, it is contemplated that, in certain embodiments, the source 252 is not so limited and may move along the entire arc length A_{L} of the intermediate body portion 256. For example, referring to FIG. 2, the source 252 may move or rotate an angle θ relative to an imaging axis I_{A} (e.g., imaging axis corresponding to the axis between the source and the detector when the source is positioned directly above the detector). In one embodiment, θ may be ±20 degrees relative to the imaging axis I_{A} so that the X-ray source 252 travels along an arc length A_{L} of the intermediate body portion 256 a full angle range of 40 degrees, although other angle ranges are contemplated based on the design of the C-arm and the SID. Thus arranged, the X-ray source can be positioned at various angles relative to the detector 254 and the imaging axis I_{A} to enable acquisition of off-axis X-ray views. This is in contrast to conventional mini C-arms where the X-ray source and detector are aligned and fixed along the imaging axis I_{A} (e.g., axis extending between the source and detector when the source is positioned directly above the detector). It should be appreciated that this is but one embodiment and other dimensions or ranges are envisioned. As illustrated, the arc length A_{L} of the intermediate body portion schematically represents the arc length that the X-ray source may travel. The arc length A_{L} is merely illustrative and not to scale.

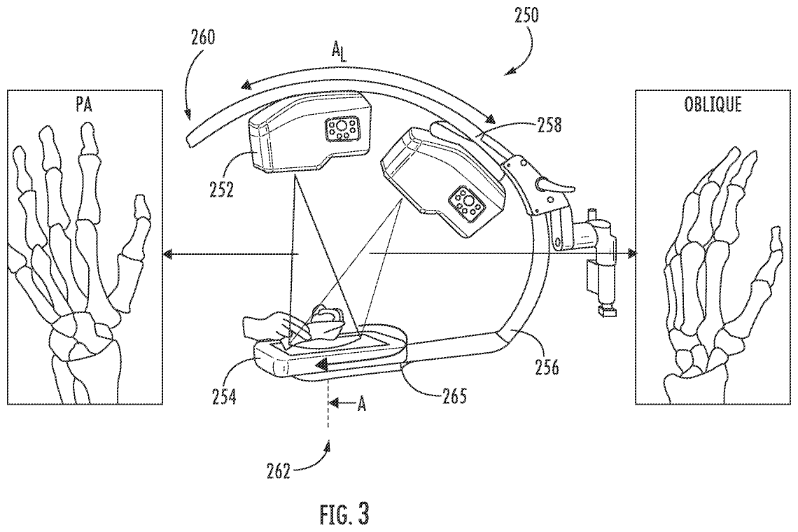

More particularly, the X-ray source 252 may be moved, repositioned, etc. to, for example, enable acquisition of multiple projection images at different angles without movement of the patient's anatomy. That is, referring to FIG. 3, the X-ray source 252 can be moved along an arc length A_{L} of the intermediate body portion 256 of the C-arm assembly 250. In moving, the X-ray source 252, the surgeon can acquire multiple projection images at different angles including, for example, an anterior-posterior view (AP), a posteroanterior view (PA), an oblique view, and/or a lateral view. In a PA view, the X-ray beam enters via the posterior (back) aspect of the patient's anatomy. The X-ray source is typically at 0 degrees to acquire the PA view. In an AP view, the X-ray beam enters via the anterior (front) aspect of the patient's anatomy. The X-ray source is typically at 0 degrees to acquire the AP view. In a lateral view, the X-ray beam (view) is substantially orthogonal to the plane that divides the patient's body into right/left halves. The X-ray source is typically at the widest angle. In an oblique view, the X-ray beam (view) is typically obtained at an angle between the lateral and AP/PA views. All of these views may be taken without moving the patient's anatomy, which may be positioned on the detector 254. As an additional benefit, the surgeon may be able to move the source 252 during the procedure to provide clearance around and access to the patient's anatomy.

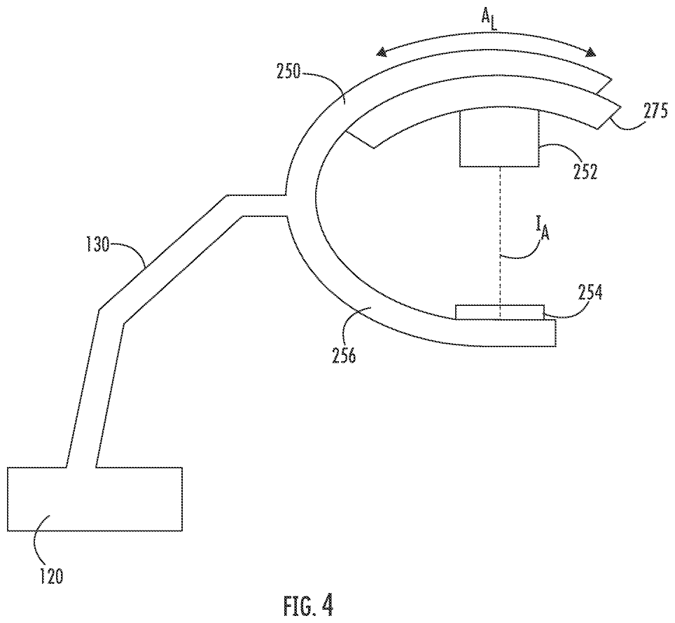

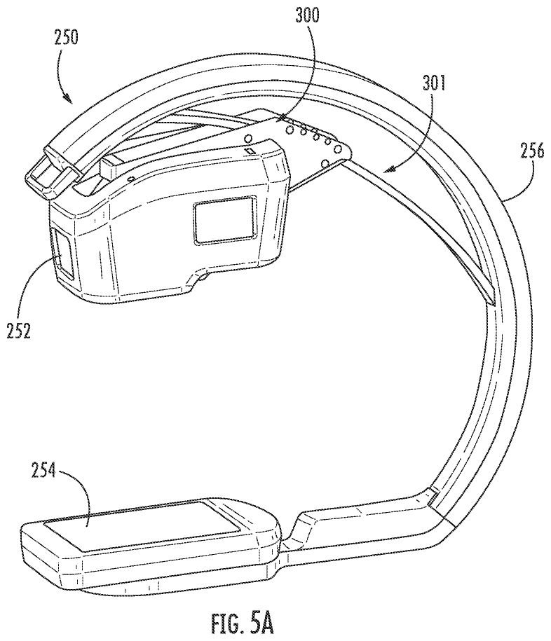

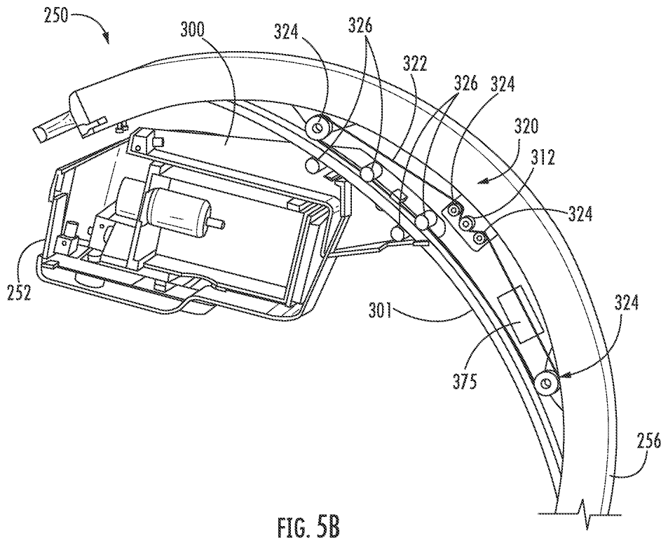

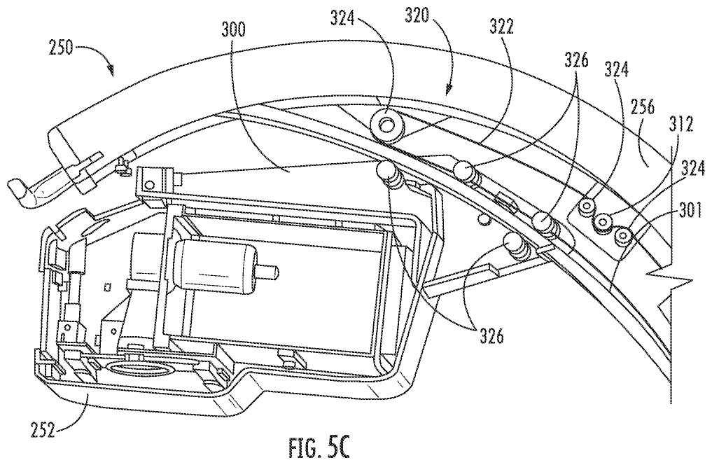

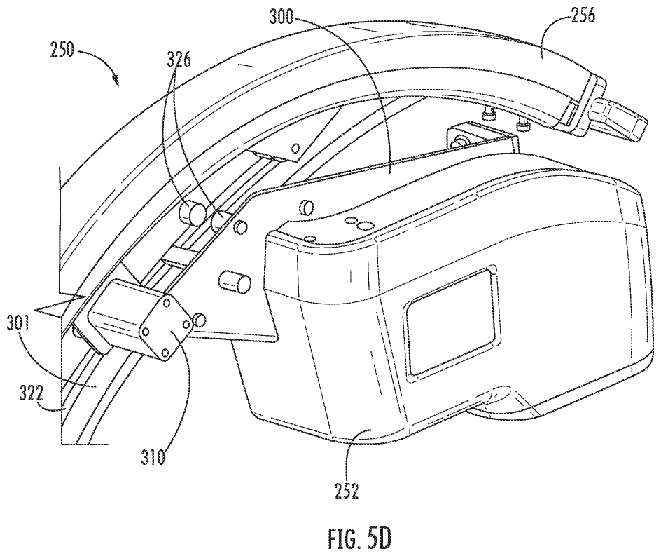

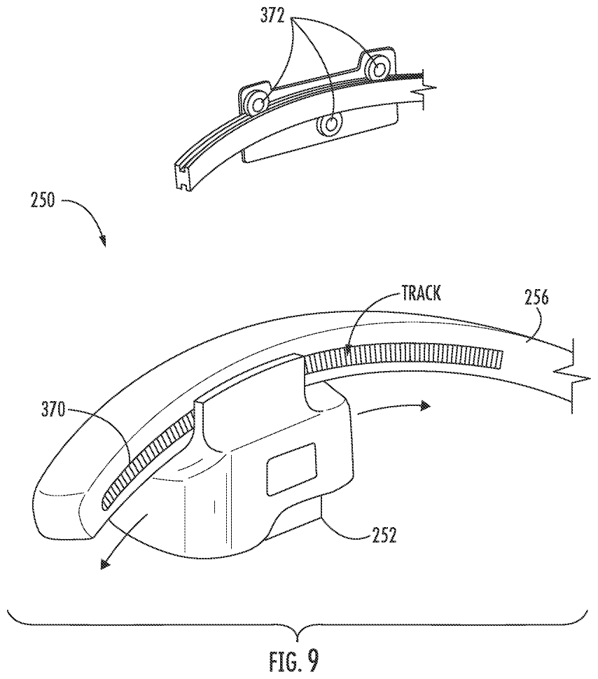

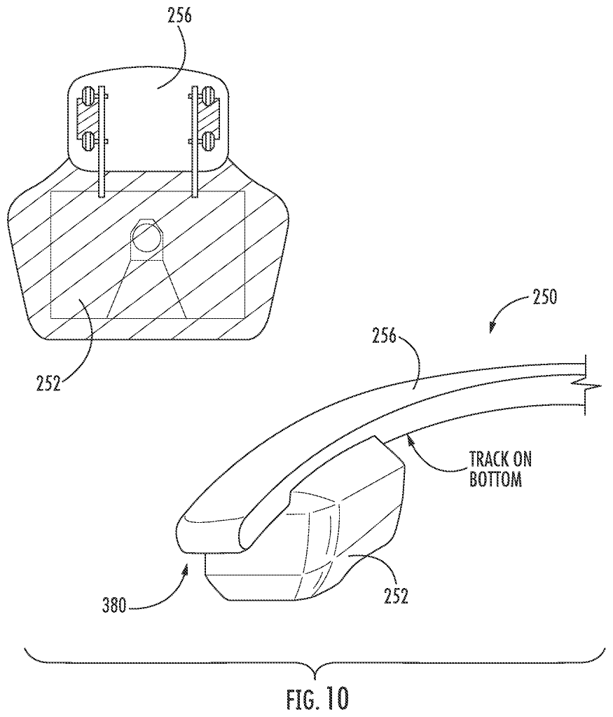

The intermediate body portion 256 of the C-arm assembly 250 may include a mechanical travel path. The mechanical travel path may comprise a track along which the X-ray source 252 may travel. In certain embodiments, the mechanical travel path or track may be provided in the form of an intermediate link 275 (FIG. 4), a rail 301 (FIGS. 5A-8), or track 370 (FIG. 9) or track 380 (FIG. 10). In addition, the X-ray source 252 may include means for coupling to and moving along the mechanical travel path (e.g., track). The track may be formed in the intermediate body portion 256 of the C-arm assembly 250 (see FIGS. 9 and 10) or comprise a separate piece attached to the intermediate body portion 256 (see FIG. 4) and rail 301 (FIGS. 5A-8). For example, the intermediate body portion 256 of the C-arm assembly 250 may include a track that extends along an arc length A_{L} thereof. As will be discussed in more detail below, the source 252 may be directly or indirectly coupled to the track so that the source 252 can be moved, repositioned, etc. along the track, which extends along the arc length A_{L} of the intermediate body portion 256 of the C-arm assembly 250.

In one embodiment, an operator can manually move the source 252 along the arc length A_{L} of the intermediate body portion 256 of the C-arm assembly 250. For example, in one embodiment, the source 252 may be coupled to the track to slide along the arc length A_{L} of the intermediate body portion 256 of the C-arm assembly 250. The source 252 and the intermediate body portion 256 of the C-arm assembly 250 may include a braking mechanism such as, for example, a spring-assisted breaking mechanism. The braking mechanism transitioning between a locked configuration and an unlocked configuration to selectively enable the operator to move the X-ray source module when in the unlocked configuration and to lock or secure a position of the X-ray source module when the motor is not in motion. In the unlocked configuration, the source 252 may be moved by the operator or via a motorized drive subsystem along the arc length A_{L} of the intermediate body portion 256 of the C-arm assembly 250. In the locked configuration, the position of the source 252 may be fixed relative to the intermediate body portion 256 of the C-arm assembly 250. The source 252 can be continuously movable along an arc length A_{L} of the intermediate body portion 256 of the C-arm assembly 250, or alternatively, the source 252 may be positionable at predefined angles, positions, etc.

Alternatively, and/or in addition, in one embodiment, the source 252 may be moved relative to the intermediate body portion 256 of the C-arm assembly 250 via, for example, motorized controls (e.g., a motorized drive subsystem). For example, the mini C-arm may include a motor to move the source 252 along an arc length A_{L} of the intermediate body portion 256 of the C-arm assembly 250. The motor may be activated via, for example, control pedals or any other control device, to activate and move the source 252 relative to the intermediate body portion 256 of the C-arm assembly 250. Alternatively, the motor can be activated by any other mechanisms now known or hereafter developed such as, for example, vocal commands, finger controls, etc. By incorporating motorized controls, movement of the source 252 can be better controlled thus facilitating precise acquisition of the various images (e.g., incorporation of motorized controls provides precise positioning of the source 252 along the arc length A_{L} of the intermediate body portion 256 of the C-arm assembly 250 to acquire images at different angles and/or positions). Thus arranged, the surgeon can generate the X-ray images from a large range of angles covering anterior-posterior views and oblique/lateral views. In addition, as will be described in greater detail below, when utilizing a mini C-arm with TOMO imaging qualities, utilization of motorized controls becomes more important since precise control of the speed and angle of the images is needed.

In certain embodiments, an intermediate link member 275 (see FIG. 4) may be coupled to the C-arm assembly and positioned along the curvature of the intermediate body portion 256. The intermediate link member 275 may form or incorporate the track discussed with reference to FIGS. 2 and 3. In one embodiment, the intermediate link member 275 may be fixedly coupled to the intermediate body portion 256 of the C-arm assembly 250. In these embodiments the intermediate link 275 may be provided as a single body, where both the link and C-arm can be fabricated as one component. In other embodiments, it is envisioned that the intermediate link member 275 may be movably coupled to the intermediate body portion 256 of the C-arm assembly 250. By incorporating an intermediate link member 275, retrofit of existing C-arm assemblies may become possible.

The X-ray source 252 may be coupled to the intermediate link member 275 and may be moveable along the length of intermediate link member 275. For example, the source 252 may include rollers to couple the source 252 to the intermediate link member 275 and to move the source 252 relative to the intermediate link member 275. The rollers may move in grooves formed in or positioned on either side of the intermediate link member 275. In other examples, there may be a motor and belt attached to the source 252 to drive movement of the source 252 relative to the intermediate link member 275. The source 252 may be movably positioned along an arc length A_{L} of the intermediate link member 275. For example, in connection with the embodiment of the C-arm assembly 250 illustrated in FIG. 4, the intermediate link member 275 extends along a curvature of the intermediate body portion 256. Thus arranged, the link member 275 and the intermediate body portion 256 can have the same arc length. In this way, the source 252 moves along the arc length of the intermediate body portion 256. Alternatively, in connection with other embodiments of the C-arm assembly 250, such as, for example, as illustrated in FIGS. 5A-5D, the intermediate link member (e.g., rail 301) is a secant line (i.e., intersects with the C-arm in two points). In this way, the source 252 moves along the rail 301, it moves through the arc length of the intermediate body portion 256 but its travel path is shorter.

As previously mentioned, in certain embodiments, the source 252 can move along a portion of the arc length A_{L} of the intermediate body portion 256. For example, referring to FIG. 2, the source 252 may move or rotate ±0 degrees of movement relative to the detector 254. In one example embodiment, θ may be equal to 20 degrees. Thus arranged, the source 252 can move ±20 degrees relative to the imaging axis I_{A} so that the X-ray source 252 can travel a full angle range of 40 degrees, although other angle ranges are contemplated based on the design of the C-arm and the SID. Alternatively, however, it is contemplated that, in certain embodiments, the source 252 is not so limited and may move along the entire arc length A_{L} of the intermediate body portion 256.

In the embodiment shown in FIGS. 5A-5D, the intermediate link member may comprise a rail 301. As will be described in greater detail herein, the rail 301 may extend along a portion of the intermediate body portion 256 of the C-arm assembly 250. The source module 252 moves or travels along a length of the rail 301. For example, as illustrated, the source module 252 may include a connector unit or housing 300 movably (e.g., slidably) coupled to the rail 301 via one or more directional alignment features discussed below. The C-arm assembly 250 may also include, or be operatively associated with, a motor 310 (FIG. 5D) operatively coupled to an output gear 312, which is operatively coupled to belt drive system 320 including a belt 322 and one or more idlers 324. During use, activation of the motor 310 rotates the output gear 312, which rotates the belt 322 about the idlers 324. Rotation of the belt 322 moves the source module 252, which may be operatively coupled to a gear for interacting with the belt 322, along the length of the rail 301.

As noted above, the source module 252 may include a directional alignment feature such as, for example, a roller slot, groove, archway, etc. As illustrated, in one embodiment, the directional alignment feature includes a plurality of rollers or bearings 326 in a frame of the connector unit 300 for interacting and guiding movement along the length of the rail 301. For example, as illustrated, the source module 252 may include a plurality of rollers or bearings 326 for interacting with the rail 301 to guide movement of the source module 252 along a length of the rail 301. As such, rotation of the motor 310 drives the belt 322 which moves the source module 252 along the rail 301. For example, activation of the motor 310 moves the source module 252 along the arc length of the rail 301 from a first or start position to a second or end position. With this arrangement, the distance between the source 252 and the detector's image plane remains constant. As noted above, the motor may be activated and controlled via, for example, control pedals or any other control device, to activate and/or rotate the output gear of the motor in a desired direction.

In addition, the mini C-arm assembly 250 may include a dynamic counterweight 375 (FIG. 5B) to enable the source module 252 to remain balanced along the arc length of the rail 301. The dynamic counterweight 375 may also aid in orbital balance of the C-arm if the C-arm lock is disengaged. Additionally, the dynamic counterweight 375 may help optimize the motor torque curve during source motion. That is, during use, the motor torque can be adjusted or changed depending on the angle or position of the X-ray source. For example, in one embodiment, the mini C-arm (e.g., firmware and software) may be configured to determine or provide a motor torque to input into the drive system based on specific positions of the X-ray source module 252 (e.g., a relative motor torque to position angle curve can be calculated and utilized). For example, with the X-ray source module 252 located at 0° position (e.g., aligned along the imaging axis with the detector 254), reduced or less torque is required to move the X-ray source module 252 as compared to moving the X-ray source module 252 when the X-ray source module 252 is positioned at the end of its range of motion. By utilizing a dynamic counterweight 375, the motor torque curve can be rendered smooth during the X-ray source modules 252 motion (e.g., the dynamic counterweight 373 can be utilized so that approximately the same amount of motor torque can be used to move the X-ray source module regardless of the position of the X-ray source module). Alternatively, in one embodiment, the imbalance may be eliminated altogether by the utilization of a dynamic counterweight. The dynamic counterweight can be configured to eliminate the imbalance caused by moving the X-ray source module along the arc travel. In use, the dynamic counterweight, is configured to move in the opposite direction of the X-ray source module to balance out the motor torque along the arc travel.

In addition, and/or alternatively, the mini C-arm may include an orbital rotation once the braking mechanism is disengaged. That is, preferably, the center of gravity of the C-arm assembly is aligned with the center of the axis of rotation. Thus arranged, the C-arm is balanced along any angle of the orbital rotation thus ensuring that the C-arm assembly does not drift once the brake mechanism is disengaged. However, in accordance with features of the present disclosure, as the X-ray source is moving during MAV/TOMO imagine acquisition, the center of gravity of the C-arm assembly may shift away from the axis of rotation thereby creating an imbalance, which may cause the C-arm assembly to drift in the orbital rotation. A dynamic counterweight can be utilized to counteract the imbalance to keep the center of gravity of the C-arm assembly from shifting.

The dynamic counterweight 375 may be a moving ballast, which is configured to move opposite to the direction of travel of the source module 252. In one example and as illustrated, the dynamic counterweight 375 is coupled to the belt 322 so that the belt 322 moves the dynamic counterweight 375 in the opposite direction of the source module 252. However, it is contemplated that the dynamic counterweight 375 may be positioned anywhere along the belt and/or idlers.

In one embodiment, the rail 301 may have a radius of approximately 22.65 inches (or 57.5 cm) centered at a center of the active area of the detector 254. Thus arranged, the X-ray source 252 can move along the arc length of the rail 301 while maintaining a 45 cm radius of movement of the focal spot of the X-ray source about the top surface of the detector 254 at the center of its active area. In one embodiment, the radius of the intermediate body portion 256 of the C-arm assembly 250 is approximately 13.37 inches (or 34 cm) to the center of the C-arm.

It should be appreciated that while motorized movement of the source 252 relative to the detector 254 has been shown and described using a belt drive system 320, other motorized and manual mechanisms may be used. For example, the motorized drive subsystem may be in the form of a lead screw, a rack & pinion, a gear train, a motorized rail, a linear actuator, etc.

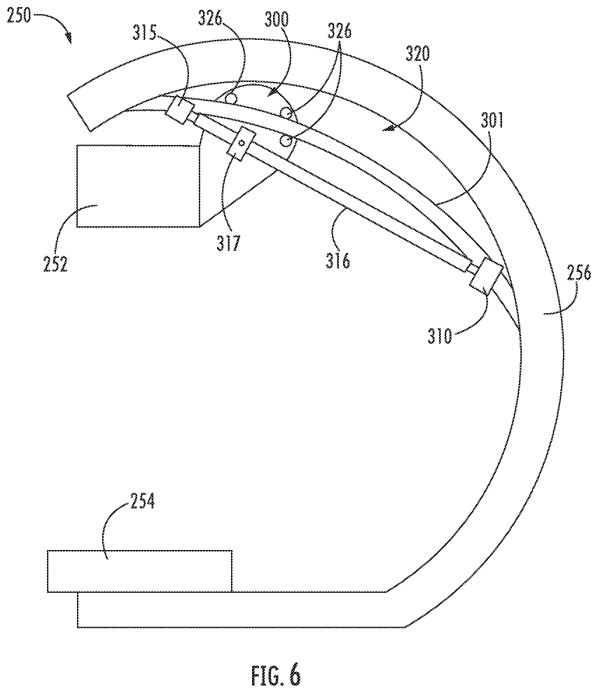

For example, referring to FIG. 6, an alternate motorized drive subsystem is shown. In use, the alternate motorized drive subsystem is substantially similar to the other embodiments disclosed herein except as described. The motorized drive subsystem 320 may utilize a motor 310 operatively coupled to a lead screw 316. That is, as illustrated, the C-arm assembly 250 may include a rail 301. The rail 301 may extend along a portion of the intermediate body portion 256 of the C-arm assembly 250. During use, the source module 252 moves or travels along a length of the rail 301. For example, as illustrated, the source module 252 may include a connector unit or housing 300 movably (e.g., slidably) coupled along a length of the rail 301. In one embodiment, the C-arm assembly 250 may also include, or be operatively associated with, a motor 310 operatively coupled to a leadscrew 316. For example, in one embodiment, the motor 310 couples, interacts with, etc. the leadscrew 316 so that activation of the motor 310 rotates the leadscrew 316. Rotation of the leadscrew 316 moves the source module 252 along the length of the rail 301.

The source module 252 may be operatively coupled to a nut (e.g., a floating leadscrew nut 317). The floating leadscrew nut 317 provides one degree of freedom to allow the leadscrew 316 to pivot relative to the source module 252 as the source module 252 moves along the length of the rail 301. As illustrated, the leadscrew 316 may also include a distal bearing 315 for coupling the leadscrew 316 to the rail 301.

Similar to other embodiments disclosed herein, the source module 252 may also include a directional alignment feature such as, for example, a roller slot, groove, archway, etc. As illustrated, in one embodiment, the directional alignment feature includes a plurality of rollers or bearings 326 in a frame of the connector unit 300 for interacting and guiding movement along the length of the rail 301. For example, as illustrated, the source module 252 may include a plurality of rollers or bearings 326 for interacting with the rail 301 to guide movement of the source module 252 along a length of the rail 301. Activation of the motor 310 turns the lead screw 316 resulting in movement of the source module 252 along the arc length of the rail 301 and relative to the detector 254 from a first or start position to a second or end position. With this arrangement, the distance between the source 252 and the detector's image plane remains constant.

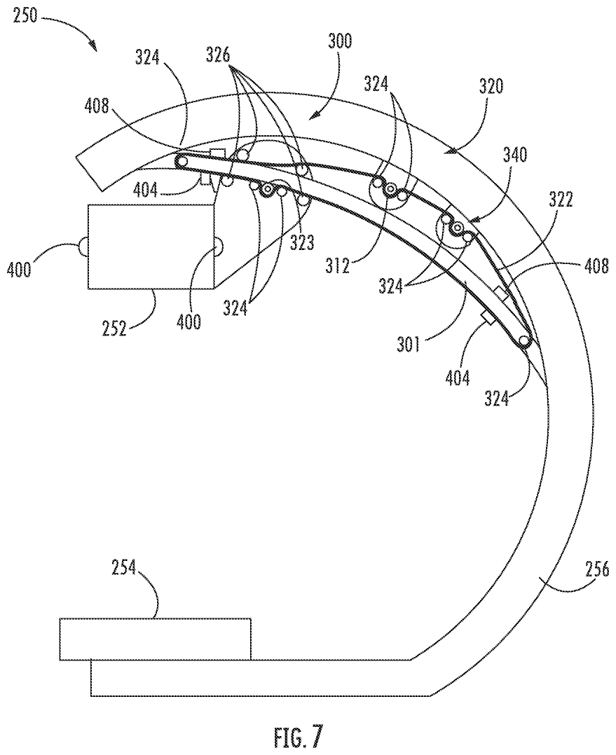

Referring to FIG. 7, an alternate motorized drive subsystem is shown. In use, the alternate motorized drive subsystem is substantially similar to the other embodiments disclosed herein except as described. The motorized drive subsystem 320 utilizes a motor 310 operatively coupled to a drive or motor belt 322. The motor 310 may include an output gear or pulley 312 operatively coupled to the drive or motor belt 322. For example, in one embodiment, the C-arm assembly 250 may include a rail 301. The rail 301 may extend along a portion of the intermediate body portion 256 of the C-arm assembly 250. The source module 252 moves or travels along a length of the rail 301. For example, as illustrated, the source module 252 may include a connector unit or housing 300 movably (e.g., slidably) coupled along a length of the rail 301. In one embodiment, the C-arm assembly 250 may also include, or be operatively associated with, a motor 310 operatively coupled to an output gear or pulley 312, which is operatively coupled to the drive or motor belt 322. In addition, the motorized drive subsystem 320 may also be operatively coupled with the connector unit 300 of the source module 252 and include a plurality of idlers 324 for adjusting the direction of the drive or motor belt 322. In one embodiment, the connector unit 300 may include a shaft with a pulley and pinion 323 for interacting with the drive or motor belt 322. During use, activation of the motor 310 rotates the output gear or pulley 312, which rotates the drive or motor belt 322 about the idlers 324. Rotation of the drive or motor belt 322 interacts with the pulley and pinion 323 to move the source module 252 along the length of the rail 301.

As previously described, the source module 252 may also include a directional alignment feature such as, for example, a roller slot, groove, archway, etc. As illustrated, in one embodiment, the directional alignment feature includes a plurality of rollers or bearings 326 in a frame of the connector unit 300 for interacting and guiding movement along the length of the rail 301. For example, as illustrated, the source module 252 may include a plurality of rollers or bearings 326 for interacting with the rail 301 to guide movement of the source module 252 along a length of the rail 301. As such, rotation of the motor 310 drives the drive or motor belt 322 which moves the source module 252 along the arc length from a first or start position to a second or end position. With this arrangement, the distance between the source 252 and the detector's image plane remains constant.

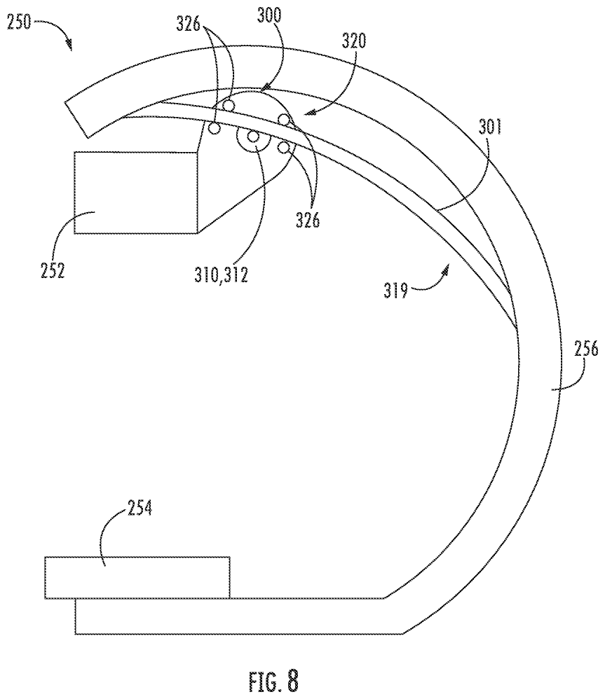

Alternatively, referring to FIG. 8, an alternate motorized drive subsystem is shown. In use, the alternate motorized drive subsystem is substantially similar to the other embodiments disclosed herein except as described. As shown, the motorized drive system 320 utilizes a motor 310 operatively coupled to the rail 301. The motor 310 may be directly coupled or associated with an output gear or pinion 312 positioned on its output shaft. Activation of the motor 310 turns the output gear or pinion 312, which moves the source module 252 along the arc length of the rail 301 and relative to the detector 254.

That is, in one embodiment, the C-arm assembly 250 may include a rail 301. The rail 301 includes a rack 319 along a surface thereof, the rack 319 interacts with the output gear or pinon 312. The rail 301 may extend along a portion of the intermediate body portion 256 of the C-arm assembly 250. The source module 252 moves or travels along a length of the rail 301. For example, as illustrated, the source module 252 may include a connector unit or housing 300 movably (e.g., slidably) coupled along a length of the rail 301. In one embodiment, the C-arm assembly 250 may also include, or be operatively associated with, the motor 310 operatively coupled to the output gear or pinon 312, which is operatively coupled to the rail 301 (e.g., rack 319). During use, activation of the motor 310 rotates the output gear or pinon 312. Rotation of the output gear or pinon 312 interacts with the rack 319 to move the source module 252 along the length of the rail 301.

As previously described, the source module 252 may also include a directional alignment feature such as, for example, a roller slot, groove, archway, etc. As illustrated, in one embodiment, the directional alignment feature includes a plurality of rollers or bearings 326 in a frame of the connector unit 300 for interacting and guiding movement along the length of the rail 301. For example, as illustrated, the source module 252 may include a plurality of rollers or bearings 326 for interacting with the rail 301 to guide movement of the source module 252 along a length of the rail 301. As such, rotation of the motor 310 rotates the output gear or pinon 312 about the rack 319 which moves the source module 252 along the arc length from a first or start position to a second or end position. With this arrangement, the distance between the source 252 and the detector's image plane remains constant.

The motorized drive subsystem may have other alternative configurations. For example, in one embodiment, the motorized drive subsystem may be in the form of a motor operatively coupled to a roller for engaging the rail. The motor may also be operatively coupled to the source module. The C-arm assembly may be operatively associated with the rail. Activation of the motor results in rotation of the rollers, which causes the source module to move along the length of the rail and thus along the arc length and relative to the detector.

In addition, the mini C-arm and/or motorized control system may include a force-assist subsystem. For example, the motorized control system may include a spring assist such as, for example, an off-the-shelf constant-force spring, which may be utilized to apply a force onto the X-ray source module during its movement. Thus arranged, the amount of force/torque that the motor needs to produce to move the X-ray source module is reduced, enabling the use of a smaller motor and reduced power/current. Alternatively, and/or in addition, a dampener such as, for example, an off-the-shelf dampener, may be utilized to prevent the X-ray source module from stopping too abruptly (e.g., to prevent or at least minimize “slamming” to a stop). The dampener slows down the motion at the end of the travel range (e.g., limits the deceleration).

Alternatively, referring back to FIG. 4, the C-arm assembly 250 may include an intermediate link member 275 positioned between the intermediate body portion 256 of the C-arm assembly 250 and the source 252. The intermediate link member 275 may be movably coupled to the intermediate body portion 256 (e.g., inner C-arm 275 may slide relative to the outer C-arm 256). In addition, the source module 252 may move relative to the intermediate link member 275 (e.g., inner C-arm). In addition, the C-arm assembly 250 may still be rotatable relative to the shifted shoe (e.g., orbital mount 170).

Referring to FIG. 9, the intermediate body portion 256 of the C-arm assembly 250 may include an arcuate or curved track 370 formed, for example, in the side surfaces thereof. The source module 252 may be operatively coupled to motorized rollers 372 coupled to the arcuate track 370. Activation of the motorized drive subsystem causes the rollers 372 of the source module 252 to move along the arcuate track 370 surface. With this arrangement, the SID, e.g., distance between the source 252 and the detector's image plane, can be configured to remain constant or variable.

Alternatively, referring to FIG. 10, the intermediate body portion 256 of the C-arm assembly 250 may include a track 380 formed, for example, in the bottom surface thereof. The source module 252 may be operatively coupled to motorized rollers coupled to the track 380. Activation of the motorized drive subsystem causes the rollers of the source module 252 to move along the track 380. With this arrangement, the SID, e.g., distance between the source 252 and the detector's image plane, can be configured to remain constant or variable.

As previously mentioned, and described, in accordance with one or more features of the present disclosure, the mini C-arm 200 may also include a motion control system. The motion control system may include a position sensing subsystem to sense, determine, etc. the position of the source module 252 along the arc length (e.g., the position sensing subsystem measures the angular position of the X-ray source module 252 relative to the detector 254 along the arc length of the mechanical travel path). The feedback from the position sensing subsystem may be used to control the movement of the X-ray source 252 as it moves along the arc length of the intermediate body portion or mechanical travel path (e.g., track or rail). In one embodiment, the position sensing subsystem may be coupled to the intermediate body portion or mechanical travel path. The position sensing subsystem may be provided in any number of suitable forms including, for example, a sensor such as, for example, a potentiometer. Alternatively, the position sensing subsystem may comprise a rotary encoder, an accelerometer, dual accelerometers, an inclinometer, a hall-effect sensor, a motor encoder, a linear inductive sensor, count pulses, any combination of gyro/accelerometer/magnetometer sensors, etc.

For example, referring to FIG. 7, the mini C-arm may include a potentiometer 340. The potentiometer 340 may be positioned in contact with or adjacent to, a moving surface such as, for example, the belt 322. Alternatively, the potentiometer 340 could be positioned in contact with the timing pulley shaft (e.g., the potentiometer could be coaxial with the shaft) or positioned within or associated with the directional alignment feature (e.g., roller slot). In one embodiment, the potentiometer may be connected to the connector unit 300. The output signal of the potentiometer 340 (e.g., resistance) correlates to an angular position of the source module 252. The firmware and/or software of the mini C-arm may include pre-defined and stored resistance values. Thereafter, by comparing the output signal of the potentiometer 340 to the pre-defined and stored resistance values, the angular position of the source module 252 can be identified. In one embodiment, the potentiometer may be co-axial to the gears or mounted to the X-ray source module and shaft and contact the rail (friction connection).

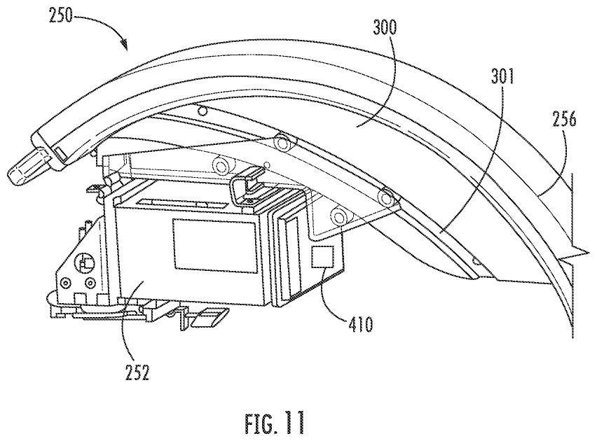

Alternatively, referring to FIG. 11, the mini C-arm may include an accelerometer 410. As illustrated, in one embodiment, the accelerometer 410 may be rigidly attached to a component of the source module 252. The output of the accelerometer 410 is used to calculate an angle (pitch and/or roll) of the source module 252.

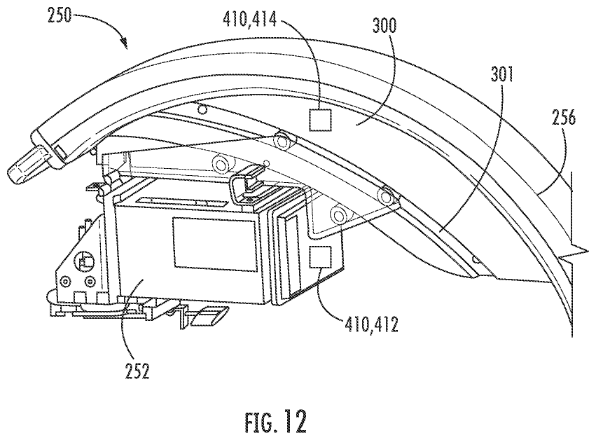

Alternatively, referring to FIG. 12, the mini C-arm may include dual accelerometers 410. As illustrated, in one embodiment, a first accelerometer 412 may be rigidly attached to a component of the source module 252. A second accelerometer 414 may be coupled to a stationary component such as, for example, the connection unit 300 between the source module 252 and the intermediate body portion 256 of the C-arm assembly 250. The output of the accelerometers 410 can be used to calculate relative displacement between the first and second accelerometers 412, 414. Based on the relative displacement, the position of the source module 252 can be calculated.

Alternatively, the position sensing subsystem may be in the form of an inclinometer such as ApexOne manufactured by Fredericks company. Alternatively, the position sensing subsystem may be in the form of a hall-effect sensor. The hall-effect sensor operates substantially similar to a potentiometer. In one embodiment, the hall-effect sensor could replace the potentiometer. For example, the hall-effect sensor could be positioned in contact with the belt. The hall-effect sensor magnet may be, for example, attached to the rotating pulley. Thus arranged, the hall-effect sensor remains stationary (e.g., does not rotate, for example, the hall-effect sensor could be positioned coaxial with the pulley shaft) and may be attached to a non-rotating surface of the X-ray source module. Rotation of the pulley causes the hall-effect sensor (angular) output to change.

Alternatively, the position sensing subsystem may be in the form of a motor encoder. The motor encoder may be attached to the motor and senses the rotational position and number of rotations of the motor's rotor.

Alternatively, the position sensing subsystem may be in the form of a linear inductive sensor. The inductive sensor may be an electrically conductive element that is in close proximity to the PCB. The inductive sensor moves linearly in relationship to this circuit. As the conductive element moves along the circuit length, the inductance changes and is converted to a displacement/position.

Alternatively, the position sensing subsystem may be in the form of a count stepper motor pluses. A motion control circuit is included to count the number of commanded motor rotation steps that it sends to the stepper motor. Since each step-pulse command results in a pre-determined angular rotation of the motor's output shaft/rotor, the angular position of the motor shaft can be determined.

In accordance with one or more features of the present disclosure, the motion control system may also include an over-travel sensing subsystem to detect and limit the maximum range of travel of the X-ray source along the arc length of the mechanical travel path. The over-travel sensing subsystem may include stops that limit travel of the X-ray source in both the clockwise (CW) and counter-clockwise (CCW) directions. In one embodiment, the stops may be software stops at programmed limits of travel from the source module's center position (e.g., ±20 degrees relative to the imaging axis I_{A}, as will be described herein). In certain embodiments, over-travel limit stops may also be provided. The over-travel limit stops may include a mechanical switch which is positioned at a slightly greater angle than the software stop angles (e.g., the mechanical switches may be positioned, for example, at ±0.5 degrees greater, thus, for example, ±20.5 degrees). The mechanical switches may halt the motor-drive signals. In all embodiments, hard stops are provided.

That is, in accordance with one or more features of the present disclosure, the over-travel sensing subsystem may be programmed with either mechanical and/or software based stops for the source 252 to avoid the C-arm assembly 250 from becoming unbalanced and/or to detect and limit the maximum range of travel of the X-ray source 252. In addition, in one embodiment, movement of the source 252 may minimize vibration of the C-arm assembly 250.

For example, the mini C-arm may include one or more stop mechanisms for controlling or limiting movement of the source 252 to prevent an unbalanced condition that could cause the mini C-arm to tip. For example, in one embodiment, the moveable base 120 may be provided with counterweights to prevent tipping of the mini C-arm as the source 252 is moved laterally or along an arc length A_{L} of the intermediate body portion 256. Alternatively, to prevent or limit lateral placement of the source 252, one or more stop mechanisms may be incorporated to limit lateral displacement of the source 252. The stop mechanisms may be any mechanisms now known or hereafter developed and may be in the form of one or more mechanical stops. Alternatively, the stops may be in the form of software which limits the movement of the source 252.

The over-travel sensing subsystem may be configured to prevent, or at least minimize, the possibility that the source 252 may be positioned in such a way that renders the mini C-arm unstable. The over-travel sensing subsystem may be any now known or hereafter developed subsystem. For example, the over-travel sensing subsystem may be or include mechanical limit switches, optical (thru-beams), proximity sensors, potentiometer (SI at limits determined during calibration), linear actuator limits, etc.

For example, with reference to FIG. 7, in one embodiment, the C-arm assembly 250 may include one or more mechanical limit switches 404. In one embodiment, the limit switches 404 may be in the form of a contact switch. During use, a mechanical surface of the X-ray source module 252 may be configured to contact the limit switches 404, the limit switch 404 being located at an over-travel limit position. Thus arranged, contact by the X-ray source module 252 with the limit switch 404 changes the open/close state of the limit switch 404, which in turn is detected by the system's motion control/sensing circuit causing movement of the X-ray source 252 to be halted.

Alternatively, in one embodiment, one or more optical thru-beam switches may be included. A non-contact switch, which may include a mechanical surface of the X-ray source module, may mechanically interfere with (e.g., break) the optical beam of a limit switch, the limit switch being located at an over-travel limit position. Thus arranged, breaking the beam changes the open/close state of the limit switch, which in turn is detected by the system's motion control/sensing circuit.

Alternatively, in one embodiment, one or more proximity sensors may be included. A non-contact switch may sense the physical distance between a mechanical surface of the X-ray source module and one or more proximity sensors. Once the sensed distance reaches a pre-defined threshold, the system's motion control/sensing circuit determines this to be an over-travel limit position. The proximity sensor may be any proximity sensor now known or hereafter developed including, for example, an inductive sensor, a capacitive sensor, an optical sensor (e.g., infrared reflectance), a magnetic sensor (e.g., a hall-effect sensor), etc.

Alternatively, in one embodiment, one or more potentiometers may be included to, for example, measure angular output. This embodiment utilizes a non-contact, indirect-sensing solution. The output values of the potentiometer at the over-travel limit positions are stored during factory/service calibration. During use, when the potentiometer output reaches a stored limit, the system's motion control/sensing circuit determines this to be an over-travel limit position.

Alternatively, in one embodiment one or more linear actuator drive systems may be included to, for example, move the X-ray source module. The actuator is (or includes) a sensor to detect a linear position of the output shaft of the actuator. The shaft positions at the over-travel limit positions are stored during factory/service calibration. Upon use, when the output reaches a stored limit, the system's motion control/sensing circuit determines this to be the over-travel limit position.

As will be appreciated by one of ordinary skill in the art, in connection with all of these embodiments, upon determination of the over-travel limit position, the mini C-arm motion control system/sensing circuitry transmits an alert such as, for example, an audible or visual alert, and/or prevent further movement of the C-arm assembly.

In addition, with reference to FIG. 7, the C-arm assembly 250 may include one or more hard stops 408. During use, the hard stops 408 may be, for example, portions of the C-arm assembly 250, which are positioned at a slightly greater angle than the over-travel limit stops (e.g., the hard stop 408 may be positioned at, for example, ±24 degrees). The hard stop 408 may mechanically halt movement of the source module 252.

In accordance with one or more features of the present disclosure, the motion control system may also include a collision-detection subsystem. The collision-detection subsystem is configured to detect and prevent the X-ray source from contacting an obstacle during its normal range of motion. In use, the collision-detection subsystem may be provided in a number of different forms. For example, the collision-detection subsystem may comprise one or more sensors which are configured to sense movement of the various components of the mini C-arm and prevent collision of the X-ray source module with an object such as, for example, a table, while the source module is being moved by the drive system. Upon sensing a collision, or a potential collision, the motor-drive signals may be halted, which in turn stops the movement of the X-ray source module. For example, with reference to FIG. 7, in one embodiment, the X-ray source 252 may include a plurality of sensors 400 thereon such as, for example, first and second sensors 400 located on a front and rear surface of the X-ray source 252. Thus arranged, the collision sensors 400 are configured to sense distance between the X-ray source 252 and any foreign obstacles. Upon detecting a potential collision, the motor-drive signals may be halted, which in turn stops the movement of the X-ray source module 252.

Alternatively, in one embodiment, the collision-detection subsystem may include an angle position/motor command (stepper) system, a sensing motor current system, a mechanical “bumper” displacement system, an accelerometer (deacceleration), a non-contact system, etc.

In one embodiment, the angle position/motor commands (stepper) collision-detection subsystem may include a drive system to enable the motor rotor to “slip” in relation to a tube module when a force higher than a pre-established threshold is applied to the tube module. This enables the tube module—and subsequently the angle sensor to stop/slow-down upon colliding with an obstacle while the motor keeps driving/rotating. In one embodiment, the subsystem may send a motor command/pulse to rotate motor, obtain an angle output value, compare pulses sent and angle value at a time stamp, and compare pulse versus angle relationship in software, firmware, or a combination thereof. If values are not synchronized within a predefined tolerance, additional movement of the mini C-arm will be prevented resulting in a halt motor command.

Alternatively, in motor current embodiment, the subsystem may monitor motor current. If a current spike and/or excessive current is detected, additional movement of the mini C-arm will be prevented resulting in a halt motor command.

Alternatively, in one embodiment the collision-detection subsystem may be in the form of a mechanical bumper system. The mechanical bumper system includes a mechanical, outboard feature to deflect upon contact with an obstacle. In one embodiment, an adjacent contact or non-contact sensor can detect the change in position of the mechanical, outboard feature upon collision and the system's motion control/sensing circuit determines this to be a collision and halts the motor signals. In some embodiments, options to fine tune/optimize the deflection force include the innate stiffness of the mechanical elements being deflected and/or the inclusion of a spring element which applies an outward force.

Alternatively, in one embodiment the collision-detection subsystem may be in the form of an accelerometer. The system may include, for example, an accelerometer in the X-ray source module. During use, the accelerometer may continuously measure the acceleration of the X-ray source module. If an “unexpected” deacceleration of the module is detected (e.g., a deacceleration which is not a result of the motion control system), the motion control circuit determines this to be a collision and halts the motor signals.

Alternatively, in one embodiment the collision-detection subsystem may be in the form of a non-contact system. The non-contact system senses (e.g., detects, monitors, etc.) the proximity of an object outboard of the system. For example, the non-contact system may include proximity sensors, laser systems, reflective systems, radar systems, etc.

It will be appreciated that while the motion control system including the positioning sensing subsystem, the over-travel sensing subsystem, and the collision-detection subsystem have been illustrated in connection with the embodiment of FIG. 7, the present disclosure is not so limited and it is envisioned that each of the embodiments disclosed herein including the embodiments of FIGS. 5, 6, and 8 may incorporate one or more features of the motion control system.

Referring to FIG. 3, in accordance with one or more features of the present disclosure that may be used in combination with movement of the source 252 along the arc length A_{L} of the intermediate body portion 256 of the C-arm assembly 250 or separate therefrom, the detector 254 rotates relative to the end portion 262 of the intermediate body portion 256 of the C-arm assembly 250. That is, the intermediate body portion 256 includes a body portion 258 and first and second end portions 260, 262 for coupling to the source and detector 252, 254, respectively. The detector 254 may be rotatable about an axis A passing through the detector 254 (e.g., as illustrated, the axis A passes perpendicular thru a front surface of the detector 254). The detector 254 may be rotatable by any mechanism now known or hereafter developed. For example, the detector may be rotatable via a rotation mechanism such as disclosed in U.S. Pat. No. 9,161,727, filed on Sep. 1, 2011, entitled Independently Rotatable Detector Plate for Medical Imaging Device, the entire contents of which are hereby incorporated by reference. When used in combination with movement of the source 252 along the arc length A_{L} of the intermediate body portion 256 of the C-arm assembly 250, rotation of the detector 254 enables additional positioning of the patient's anatomy to facilitate acquisition of AP or PA views without movement of the patient's anatomy.

The detector 254 may rotate by any mechanism now known or hereafter developed. For example, the detector 254 may be positioned within a housing 265, the housing 265 being rotatably coupled to the end portion 262 of the intermediate body portion 256 of the C-arm assembly 250.

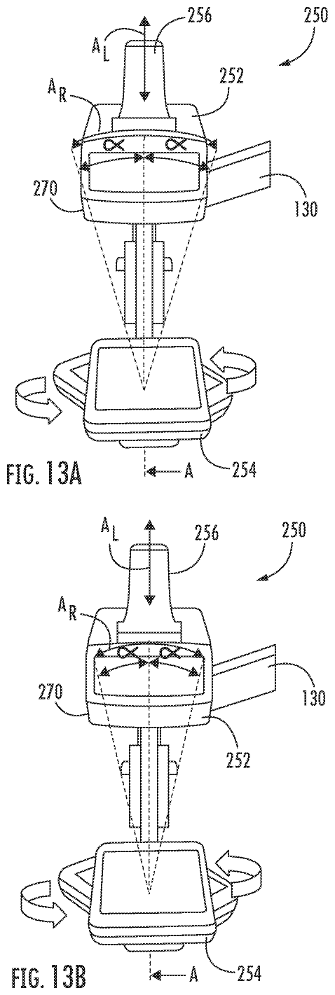

Referring to FIGS. 13A and 13B, in accordance with one or more features of the present disclosure that may be used in combination with movement of the source 252 along the arc length A_{L} of the intermediate body portion 256 of the C-arm assembly 250 and/or the rotatable detector 254, or separate therefrom, the source 252 may move along an arc Aᵣ that is substantially perpendicular to an arc length A_{L} of the intermediate body portion 256 of the C-arm assembly 250. The source 252 may be movable along an arc Aᵣ that is substantially perpendicular to an arc length A_{L} of the intermediate body portion 256 of the C-arm assembly 250 by any mechanism now known or hereafter developed. For example, referring to FIG. 13A, the X-ray source 252 may be positioned within a source housing 270. The source housing 270 and the X-ray source 252 may be movable along arc Aᵣ. Alternatively, referring to FIG. 13B, the X-ray source 252 may be movable within the source housing 270. Thus arranged, the operator does not see the motion of the x-ray source 252 and it does not affect the surgery since the source housing 270 remains stationary. Alternatively, in one embodiment, the X-ray tube may move along arc Aᵣ within the source housing 270. In either implementation, the X-ray source 252 may be moved in either direction by angle α thereby enabling movement of the source 252 relative to the detector 254. In one embodiment, a may be 15 degrees so that the source 252 may provide ±15 degrees of movement along an arc Aᵣ that is substantially perpendicular to an arc length A_{L} of the intermediate body portion 256 of the C-arm assembly 250.

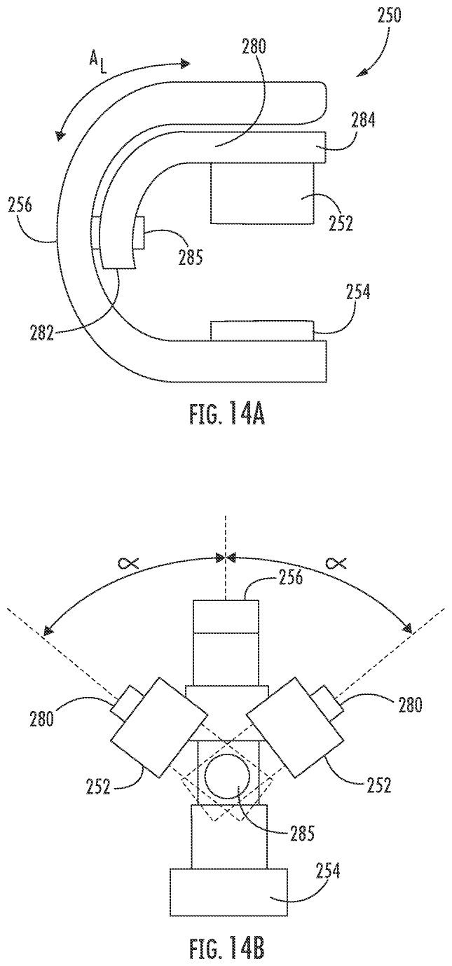

Alternatively, referring to FIGS. 14A and 14B, the source 252 may be positioned on a secondary link member 280. For example, the secondary link member 280 may include a first end 282 and a second end 284. The first end 282 of the secondary link member 280 may be coupled to intermediate body portion 256 of the C-arm assembly 250. For example, the first end 282 of the secondary link member 280 may be coupled via a rotatable pin mechanism 285. As illustrated, the first end 282 of the secondary link member 280 may be positioned in a central portion of the intermediate body portion 256 of the C-arm assembly 250. The secondary link member 280 may be rotated in either direction by angle α thereby enabling movement of the source 252, which is coupled to the second end 284 of the secondary link member 280, to facilitate movement of the source 252 relative to the detector 254. In one embodiment, a may be 20 degrees so that the source 252 may provide ±20 degrees of movement along an arc Aᵣ that is substantially perpendicular to an arc length A_{L} of the intermediate body portion 256 of the C-arm assembly 250. In connection with the current embodiment, by utilizing a secondary link member to couple the source 252 to the C-arm assembly 250, the distance between the source 252 and the detector's image plane can vary.

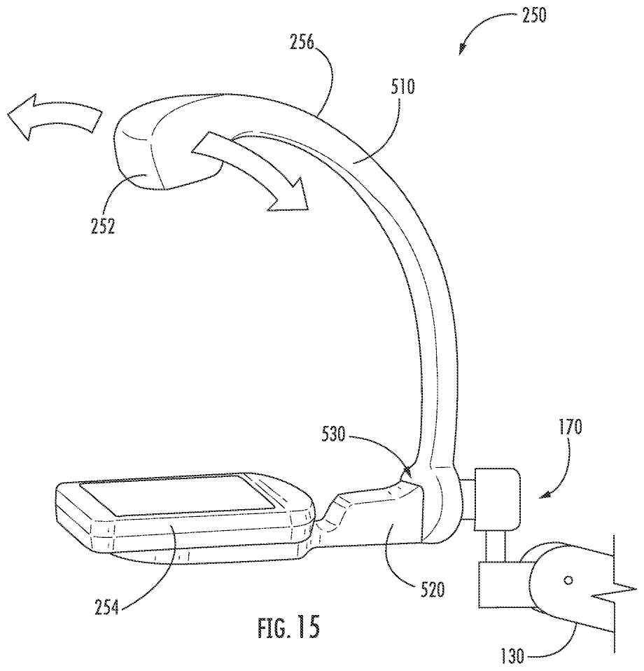

Referring to FIG. 15, an alternate embodiment of a C-arm assembly 250 for enabling lateral movement of the source 252 relative to the detector 254 is illustrated. In the alternate embodiment shown, the intermediate body portion 256 of the C-arm assembly 250 may be manufactured from first and second segments 510, 520 coupled together. The first segment 510 may include the source 252. The second segment 520 may include the detector 254. The first segment 510 may be pivotably coupled to the second segment 520 so that the source 252 is pivotably coupled to the detector 254. In one embodiment, as illustrated, the second segment 520 may be substantially straight and may include the detector 254 coupled to a first end thereof while the first segment 510 may be pivotably coupled to the second segment 520 at a second end thereof opposite of the detector 254. Thus arranged, the pivot point 530 may be substantially aligned with the image plane of the detector 254. In addition, thus arranged, the distance between the source 252 and the detector 254 remains constant. The first and second segments 510, 520 may be pivotably coupled to each other by any mechanism now known or hereafter developed including any mechanisms disclosed herein.

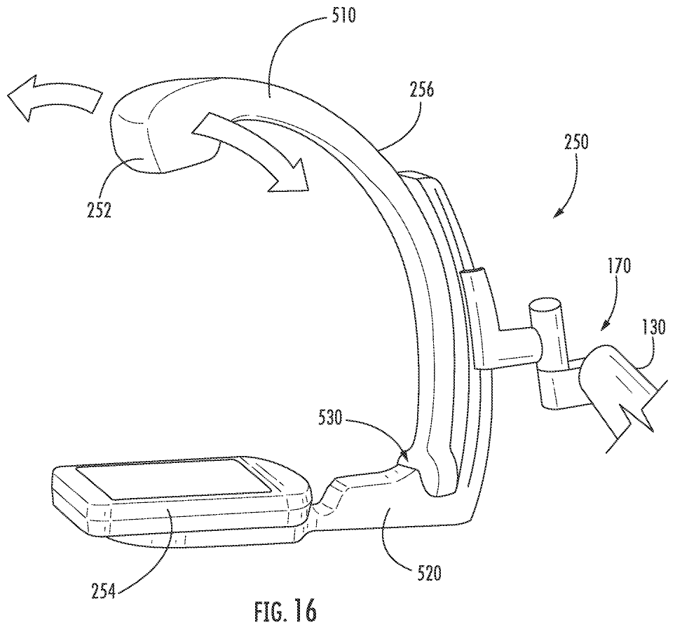

Referring to FIG. 16, an alternate embodiment of a C-arm assembly 250 for enabling lateral movement of the source 252 relative to the detector 254 is illustrated. The alternate embodiment is substantially similar to the embodiment described above in connection with FIG. 15 except as described herein. In the alternate embodiment shown, the second segment 520 associated with the detector 254 may include an approximate L-shape so that the second segment 520 may be operatively coupled with the orbital mount 170 of the C-arm assembly 250 to maintain rotation movement of the C-arm assembly 250. Thus arranged, with the second segment 520 rotationally coupled to the C-arm assembly 250 and with the second segment 520 pivotably coupled to the first segment 510, the source 252 may be pivotably coupled to the detector 254 while still enabling rotationally movement of the C-arm assembly 250 relative to the arm assembly 130. In addition, as with the embodiment of FIG. 15, the pivot point 530 between the first and second segments 510, 520 coincides with the image plane of the detector 524. In addition, thus arranged, the distance between the source 252 and the detector 524 remains constant. The first and second segments 510, 520 may be pivotably coupled to each other by any mechanism now known or hereafter developed including any mechanisms disclosed herein.

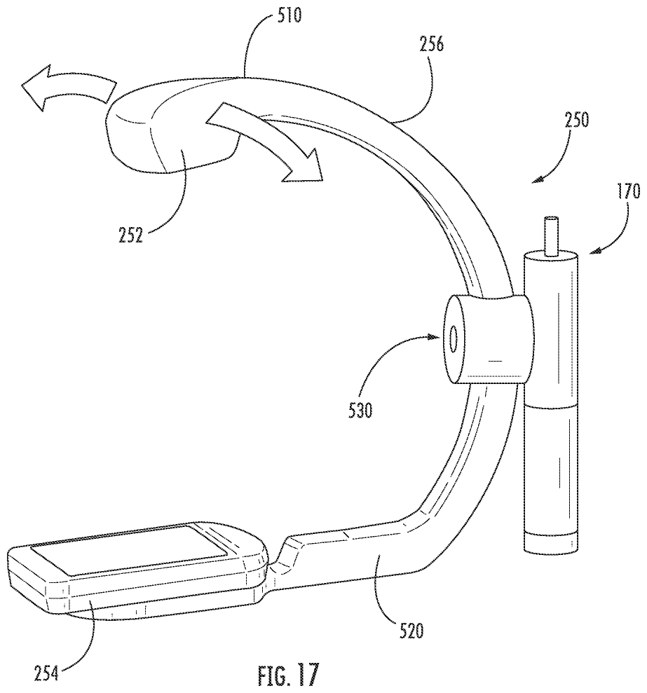

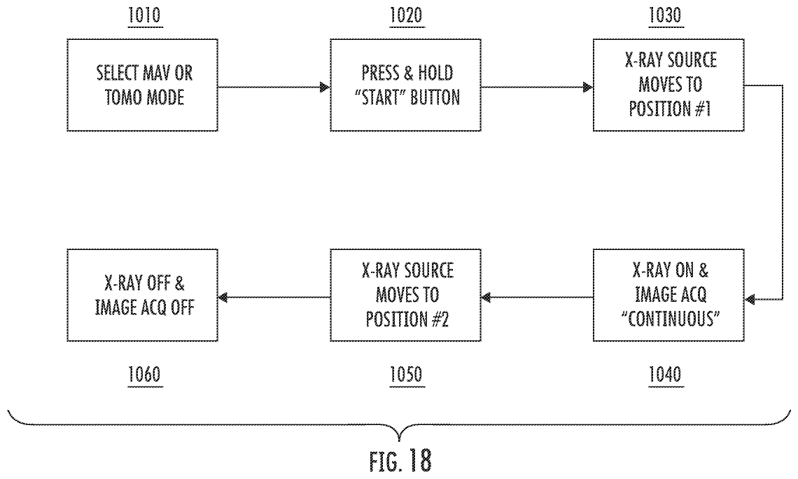

Referring to FIG. 17, another alternate embodiment of a C-arm assembly 250 for enabling lateral movement of the source 252 relative to the detector 254 is illustrated. The alternate embodiment is substantially similar to the embodiment described above in connection with FIG. 15 except as described herein. In the alternate embodiment shown, the first segment 510 of the intermediate body member 256 may be pivotably coupled to the second segment 520 of the intermediate body member 256 at a midpoint thereof. Thus arranged, by positioning the pivot point 530 substantially approximate to the horizontal centerline of the C-arm assembly 250, the distance between the source 252 and the detector 254 can vary.

By enabling the source 252 to move along an arc Aᵣ that is substantially perpendicular to an arc length A_{L} of the intermediate body portion 256 of the C-arm assembly 250, TOMO imaging acquisition may be implemented into a mini C-arm. That is, the X-ray source 252 may be moved over the patient's anatomy while taking multiple images in seconds. Thereafter, the images can be combined to generate a 3D image or volume of the patient's anatomy. As will be appreciated by one of ordinary skill in the art, TOMO utilizes acquisition of multiple images while the source 252 moves along and/or across the patient's anatomy. Thereafter, the images may be inputted into a computerized system that creates a 3D image or volume of the patient's anatomy based on the generated images. In addition, and/or alternatively, the source 252 may be moved to, for example, create a larger working space (e.g., surgeons have the ability to move the source 252 out of their way as desired). In addition, and/or alternatively, the source 252 and the detector 254 may be used to acquire multiple images of the patient's anatomy. These images may be used to generate multiple images at various angles of the patient's anatomy.

In addition, in accordance with one or more features of the present disclosure and as previously mentioned, the source 252 moves relative to the detector 254 and/or relative to the intermediate body portion 256 of the C-arm 250 via a manual operation (e.g., an operator can manual move the source 252) or via motorized controls (e.g., C-arm assembly 250 may include one or more motors to move the source 252). In one implementation, when performing TOMO to generate a 3D image or volume of the patient's anatomy, motorized control of the source 252 along an arc length A_{L} of the intermediate body portion 256, along an arc Aᵣ perpendicular to the arc length A_{L} of the intermediate body portion 256, and/or rotation of the detector 254 about axis A is preferred since generation of a 3D image or volume requires precise control over the positioning of the source 252 for each individual image.

In addition, and/or alternatively, it is envisioned that the mini C-arm may be programmable so that individual surgeons can preprogram pre-set angles and/or positions for the source 252 to meet operator preferences.

As previously mentioned herein, in accordance with one or more features of the present disclosure, by enabling the source 252 to be movable relative to the detector 254 during image capture, the mini C-arm enables MAV and/or TOMO image acquisition.

For example, referring to FIG. 18, an example embodiment of a MAV and/or TOMO image acquisition method is disclosed. In accordance with one or more features of the image acquisition method, the method may be used to acquire multiple images at different positions and/or angles regardless if MAV or TOMO imaging is being utilized. That is, substantially the same process or method may be used by the operator to acquire multiple images. As such, a more efficient workflow is provided for the operator.

As will be described herein, the image acquisition method may be used to continuously acquire images throughout a range of angles or positions of the X-ray source relative to the detector. That is, the X-ray source may be initially activated and the X-ray source may be moved between various positions such as, for example, first and second positions (e.g., X-ray source is continuously ON as the X-ray source moves between the first and second positions, thus creating a series of images at different angles between the first and second positions). As a result, as the X-ray source moves along the arc length of the curved intermediate body portion of the C-arm assembly relative to the detector, a plurality of projection images of the patient's anatomy are acquired without moving the patient's anatomy from a surface of the detector. In one embodiment, the images are continuously acquired as the X-ray source moves between the first and second positions. In addition, in one embodiment, the X-ray source automatically moves between the first and second positions. In certain embodiments, the first and second positions correspond to predetermined positions, pre-selected by the operator to acquire desired images.

Thereafter, depending on whether MAV or TOMO imaging is being utilized, the processing of the plurality of images post-acquisition and the display of the images may differ between the two modes. For example, in connection with MAV, the images may be displayed side-by-side illustrating two separate 2D images acquired at different angles. In one embodiment, the displayed images includes a first image acquired at the first position and a second image acquired at the second position. Alternatively, the displayed images includes first and second images selected by the operator from the plurality of projection images acquired as the X-ray source moves between the first and second positions.

Meanwhile, with TOMO, a 3D reconstructed image may be generated and then displayed (e.g., a three-dimensional reconstruction of the patient's anatomy using the plurality of projection images may be generated). Both MAV and TOMO may also display the full sequence of images acquired (e.g., 2D Cine-type image). This enables the operator to select the images to be displayed (e.g., show images from the first position and the second position or a movie of all of the images acquired between the first and second positions). In addition, in one embodiment, a sequence of all of the projection images acquired as the X-ray source moves between the first position and the second position may be displayed as, for example, a movie or video.

Referring to FIG. 18, the MAV and/or TOMO image acquisition method may include, at step 1010, selecting MAV or TOMO mode. For example, in one embodiment, the user may elect the desired operational mode by pressing an image acquisition selection mode, although any other now known or hereafter developed mechanisms for selecting between MAV and TOMO imagine acquisition modes may be used. Alternatively, it is envisioned that the selection of MAV or TOMO modes of operation may be selected post-image acquisition.

Next, at step 1020, after selecting the desired image acquisition mode (e.g., MAV or TOMO), the user may initiate the image acquisition. For example, the user may press and hold an X-ray ON button to initiate image acquisition and turn ON the X-ray source, although any other now known or hereafter developed mechanisms for starting the mini C-arm and/or X-ray source may be used.

At step 1030, the X-ray source is moved to a first or start position and/or angle. Alternatively, it is envisioned that the X-ray source may be initially moved to a first or start position and/or angle and then the mini C-arm and/or X-ray source may be activated. In either event, the first or start position and/or angle may be a pre-set position and/or angle, or may be set via a user command (e.g., not a pre-set position and/or angle).

At step 1040, with the X-ray source ON, the mini C-arm may begin to acquire a first image.

At step 1050, the X-ray source is moved to a second position and/or angle. The second position and/or angle may be a pre-set position and/or angle, or may be via a user command (e.g., not a pre-set position and/or angle). As previously mentioned, in one embodiment, the X-ray source remains continuously ON as the X-ray source moves between the first and second positions thus enabling a plurality of images of the patient's anatomy to be acquired as the X-ray source moves between the first and second positions.

At step 1060, the mini C-arm and/or image acquisition may be turned OFF. For example, in one embodiment, the X-ray source may be turned off automatically upon the user releasing the X-ray ON button. Upon completion, the image, angle, time stamp data, etc. may be sent to a GPU for image processing.

During the disclosed workflow, the user presses and holds the X-ray ON button throughout the whole workflow, but the X-ray source turns ON automatically once the start position is reached and shut off automatically once the end position is reached. This helps to prevent overexposure of the operator and the patient. It is envisioned that alternate automatic exposure control devices and/or mechanisms may be used.

In certain other embodiments, MAV image acquisition and TOMO image acquisition may be either via a continuous mode or a snapshot mode. In both scenarios, the method of acquiring a MAV or TOMO image is substantially the same. The primary difference being the duration or time that the X-ray source energy remains on. In continuous mode, the X-ray source energy may remain on while the user continuously holds down the X-ray ON switches and, upon releasing the switch, a still image is acquired. In snapshot image acquisition mode, the X-ray source energy may be automatically turned off by the device once the device determines that an image of acceptable image quality has been acquired. Similar to the continuous mode, a still image is acquired. In either event (continuous or snapshot) the movement of the X-ray source may be decoupled from the acquisition of the images.

In certain embodiments, the mini C-arm may also include a collimator/field of view (FOV) control subsystem to collimate the beam to match the detector active area as the source moves. For example, in one embodiment, the collimator/field of view (FOV) control subsystem may control the collimator's aperture size and position while the X-ray source module travels through its full range of motion.

Alternatively, in one embodiment, the mini C-arm may enable the user to select a custom size and position of the FOV. In one embodiment, a one-step sequence may be utilized. The position to region of interest may be determined, with user FOV size and placement input via touchscreen. Mag-view enables a reduced dose (due to reduced aperture size), and increasing exposure is an option for improved image quality. During use, the laser should be turned OFF during Mag View.

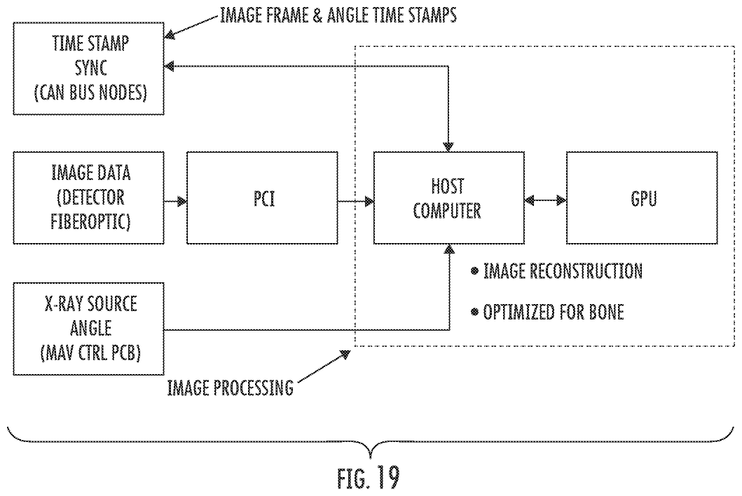

Image processing may be performed by any methods now known or hereafter developed. For example, in one embodiment, referring to FIG. 19, image processing may include acquiring image raw data and acquiring the angular position of the X-ray source for every image acquired. The angular position is recorded whenever a command is sent to acquire an image, and this angle-image “pair” enables image reconstruction of, for example, the TOMO images. For example, as illustrated, the mini C-arm may include, or be operatively associated with, various subsystems for collecting the image raw data, the angle or position of the X-ray source for each of the collected images, and for time stamping each of the collected images. The information may then be provided to an image processing subsystem including a host computer and a graphics card, the image processing subsystem collects the image raw data, the X-ray source angle, and the time stamp data. The image processing subsystem reconstructing the collected data into one or more images as described herein.

That is, during image acquisition, the angular position of each of the acquired images is recorded to facilitate image processing. For example, during TOMO image acquisition, information about the source angle for each X-ray may be used to reconstruct the three-dimensional image. Thus, in addition to controlling the motion of the X-ray source, the acquisition of images by the detector should be coordinated as the X-ray source moves through its range of travel, and the subsequent processing of these images should be delivered to the end user.

In addition, and/or alternatively, the mini C-arm may include a C-arm balance subsystem. The C-arm balance subsystem may be any subsystem now known or hereafter to balance the C-arm during movement of the X-ray source module. For example, the C-arm balance subsystem may be a counterweight on the C-arm extrusion extension, a counterweight on the shifted shoe extension, a counterweight on a linkage, a counterweight on the drive belt, locks on the raygun and shifted shoe, electronic locks and confirmation, etc.

In addition, and/or alternatively, the mini C-arm may include a flex-arm balance subsystem. The flex-arm balance subsystem may be any subsystem now known or hereafter to balance the flex-arm during the movement of the X-ray source module. For example, the flex-arm balance subsystem may be a manual lock, an electro-mechanical lock, a gas-spring, etc. In one embodiment, the gas spring may handle a maximum load.

The source 252 and the detector 254 may be any source and detector now known or hereafter developed. For example, the X-ray source module 252 may include an X-ray source, a housing or enclosure, a control panel, for example, mounted on the housing and facing the user for accessibility, a collimator attached to the X-ray source, a laser attached to the collimator or X-ray source, a detector illumination attached to the collimator or X-ray source, and control PCBs positioned, for example, inside of the housing. The detector 254 may be, for example, a flat panel detector including, but not limited to, an amorphous silicon detector, an amorphous selenium detector, a plasma-based detector, etc. The source 252 and detector 254 create an image of a patient's anatomy, such as for example a hand, a wrist, an elbow, a foot, etc.

While the present disclosure makes reference to certain embodiments, numerous modifications, alterations, and changes to the described embodiments are possible without departing from the sphere and scope of the present disclosure, as defined in the appended claim(s). Accordingly, it is intended that the present disclosure not be limited to the described embodiments, but that it has the full scope defined by the language of the following claims, and equivalents thereof. The discussion of any embodiment is meant only to be explanatory and is not intended to suggest that the scope of the disclosure, including the claims, is limited to these embodiments. In other words, while illustrative embodiments of the disclosure have been described in detail herein, it is to be understood that the inventive concepts may be otherwise variously embodied and employed, and that the appended claims are intended to be construed to include such variations, except as limited by the prior art.

The foregoing discussion has been presented for purposes of illustration and description and is not intended to limit the disclosure to the form or forms disclosed herein. For example, various features of the disclosure are grouped together in one or more embodiments or configurations for the purpose of streamlining the disclosure. However, it should be understood that various features of the embodiments or configurations of the disclosure may be combined in alternate embodiments or configurations. Moreover, the following claims are hereby incorporated into this Detailed Description by this reference, with each claim standing on its own as a separate embodiment of the present disclosure.

As used herein, an element or step recited in the singular and proceeded with the word “a” or “an” should be understood as not excluding plural elements or steps, unless such exclusion is explicitly recited. Furthermore, references to “one embodiment” of the present disclosure are not intended to be interpreted as excluding the existence of additional embodiments that also incorporate the recited features.

The phrases “at least one”, “one or more”, and “and/or”, as used herein, are open-ended expressions that are both conjunctive and disjunctive in operation. The terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure. Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. All rotational references describe relative movement between the various elements. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

What is claimed is:

1. A mini C-arm imaging apparatus comprising:
a C-arm assembly;
a movable base; and
an arm assembly coupling the C-arm assembly to the movable base;
wherein the C-arm assembly includes:
a first end, a second end, and a curved intermediate body portion extending between the first and second ends, the C-arm assembly including an X-ray source adjacent the first end and a detector at the second end, the curved intermediate body portion defines an arc length extending between the first and second ends, the X-ray source being moveable along the arc length of the curved intermediate body portion and relative to the detector to enable the mini C-arm to acquire a first image when the X-ray source is at a first position on the curved intermediate body portion and a second image when the X-ray source is at a second position on the curved intermediate body portion, the second position being different that the first position, so that the first and second images of the patient's anatomy are taken at different angles relative to the patient's anatomy and are acquired without moving the patient's anatomy during a surgical procedure.

2. The mini C-arm imaging apparatus of claim 1, wherein the curved intermediate body portion of the C-arm assembly includes a rail, the X-ray source being movably coupled to the rail.

3. The mini C-arm imaging apparatus of claim 2, wherein the X-ray source is manually movable along a length of the rail.

4. The mini C-arm imaging apparatus of claim 2, wherein the X-ray source is moved along a length of the rail via a drive system.

5. The mini C-arm imaging apparatus of claim 4, wherein the drive system includes a motor operatively coupled to a belt and one or more idlers, and wherein activation of the motor rotates the belt about the one or more idlers to move the X-ray source along the length of the rail.

6. The mini C-arm imaging apparatus of claim 5, wherein the X-ray source includes a connector unit movably coupled to the rail and a directional alignment feature for guiding movement along the length of the rail.

7. The mini C-arm imaging apparatus of claim 5, further comprising a dynamic counterweight to balance the X-ray source as the X-ray source moves along the length of the rail.

8. The mini C-arm imaging apparatus of claim 1, wherein the C-arm assembly further comprises an intermediate link member coupled to the curved intermediate body portion adjacent the first end of the C-arm assembly, wherein the X-ray source is movable coupled to the intermediate link member to position the X-ray source along the arc length of the curved intermediate body portion.

9. The mini C-arm imaging apparatus of claim 8, wherein the intermediate link member is fixed to the C-arm assembly.

10. The mini C-arm imaging apparatus of claim 8, wherein the intermediate link member is movably coupled to the C-arm assembly.

11. The mini C-arm imaging apparatus of claim 1, wherein the X-ray source moves ±20 degrees along the arc length of the curved intermediate body portion of the C-arm assembly and relative to an axis passing through the X-ray source and the detector when the X-ray source is positioned directly above the detector.

12. The mini C-arm imaging apparatus of claim 1, wherein the detector is rotatable about an axis passing through the X-ray source and the detector when the X-ray source is positioned directly above the detector.

13. The mini C-arm imaging apparatus of claim 12, wherein the detector is positioned within a housing, the housing is rotatably coupled to the second end of the curved intermediate body portion of the C-arm assembly.

14. The mini C-arm imaging apparatus of claim 1, wherein the X-ray source is movable along an arc extending perpendicular to the arc length of the curved intermediate body portion of the C-arm assembly.

15. The mini C-arm imaging apparatus of claim 14, wherein the X-ray source is positioned within a source housing, the source housing and the X-ray source are movable relative to the detector along the arc extending perpendicular to the arc length of the curved intermediate body portion of the of the C-arm assembly.

16. The mini C-arm imaging apparatus of claim 14, wherein the X-ray source is positioned within a source housing, the X-ray source is movable relative to the source housing and the detector along the arc extending perpendicular to the arc length of the curved intermediate body portion of the of the C-arm assembly.

17. The mini C-arm imaging apparatus of claim 14, further comprising a secondary link member, the secondary link member includes a first end rotatably coupled to the C-arm assembly and a second end coupled to the X-ray source, the secondary link member being rotatable relative to the C-arm assembly so that the X-ray source moves along the arc extending perpendicular to the arc length of the curved intermediate body portion of the of the C-arm assembly.

18. A mini C-arm imaging apparatus comprising:
a C-arm assembly;
a movable base; and
an arm assembly coupling the C-arm assembly to the movable base;
wherein the C-arm assembly includes:
a first end, a second end, a curved intermediate body portion extending between the first and second ends, and a rail coupled to the C-arm assembly and extending between portions of the curved intermediate body portion of the C-arm assembly, the rail defining an arc length;
an X-ray source movably coupled to the rail;
a detector at the second end of the C-arm assembly; and
a drive system associated with the X-ray source, the drive system including a motor operatively coupled to a belt and one or more idlers, wherein activation of the motor rotates the belt about the one or more idlers to move the X-ray source along the arc length of the rail.

19. The mini C-arm imaging apparatus of claim 18, wherein the X-ray source is movable along the arc length of the rail to enable the mini C-arm to acquire a first image at a first position along the curved intermediate portion and a second image at a second position along the curved intermediate portion, the second position being different that the first position so that first and second images of the patient's anatomy are taken at different angles and are acquired without moving the patient's anatomy during a surgical procedure.

20. The mini C-arm imaging apparatus of claim 18, wherein the X-ray source includes a connector unit movably coupled to the rail and a directional alignment feature for guiding movement along the arc length of the rail.

21. The mini C-arm imaging apparatus of claim 18, wherein the X-ray source provides ±20 degrees of movement relative to the detector and an imaging axis along the arc length of the rail, the imaging axis being defined as the axis passing through the X-ray source and the detector when the X-ray source is positioned directly above the detector.

22. The mini C-arm imaging apparatus of claim 18, wherein the detector is rotatable about an axis passing perpendicular to a surface of the detector.

23. The mini C-arm imaging apparatus of claim 18, further comprising a motion control system to control movement of the x-ray source along the arc length of the rail.

24. A method of acquiring multiple images using a mini C-arm including a C-arm assembly having a first end, a second end, a curved intermediate body portion extending between the first and second ends, the mini C-arm including an X-ray source moveable along an arc length of the curved intermediate body portion of the C-arm assembly and a detector positioned at the second end of the C-arm assembly, the method comprising:
moving the X-ray source along the arc length of the curved intermediate body portion of the C-arm assembly relative to the detector between a first position on the curved intermediate body portion and a second position on the curved intermediate body portion; and
acquiring a plurality of projection images of a patient's anatomy without moving the patient's anatomy from a surface of the detector as the x-ray source moves between the first and second positions.

25. The method of claim 24, further comprising displaying two or more projection images on a display device.

26. The method of claim 25, wherein the step of displaying the two or more projection images includes displaying the projection image acquired at the first position and the projection image acquired at the second position.

27. The method of claim 25, wherein the step of displaying the two or more projection images includes the step of selecting at least two projection images from the plurality of projection images acquired as the X-ray source moves between the first and second positions.

28. The method of claim 25, further comprising displaying the two or more projection images with a video of all of the plurality of projection images acquired as the X-ray source moves between the first position and the second position.

29. The method of claim 24, further comprising generating a three-dimensional reconstruction of the patient's anatomy using the plurality of projection images.

30. The method of claim 29, further comprising displaying the three-dimensional reconstruction of the patient's anatomy.

31. The method of claim 24, further comprising selecting one of multi-angle view (MAV) imagine acquisition mode or tomosynthesis (TOMO) image acquisition mode before acquiring the plurality of projection images; and processing the plurality of projection images for display on a display device based on the selected mode.

32. The method of claim 24, wherein images are continuously acquired as the X-ray source moves between the first and second positions.

33. The method of claim 24, wherein the X-ray source automatically moves between
the first and second positions.
